(19) United States
(12) Patent Application Publication
(10) Pub. No.: US 2003/0095957 A1
(43) Pub. Date: May 22, 2003

(54) Activation of dendritic cells to enhance immunity

(75) Inventors: Ronald G. Crystal, New York, NY (US); Toshiaki Kikuchi, Sendai (JP); Toshiaki Fushimi, New York, NY (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cornell Research Foundation, Inc., Ithaca, NY (US)

(21) Appl. No.: 10/320,140

(22) Filed: Dec. 16, 2002

Related U.S. Application Data
(63) Continuation of application No. 09/586,702, filed on Jun. 1, 2000.
(60) Provisional application No. 60/137,042, filed on Jun. 1, 1999.

(51) Int. Cl. A61K 48/00; C12N 15/861

(52) U.S. Cl. 424/93.21; 435/456

(57) ABSTRACT

The present invention provides a method of enhancing immunity in a mammal. The method comprises modifying a dendritic cell (DC) in vivo or ex vivo to produce a dendritic cell-mediator in the mammal. The dendritic cell-mediator up-regulates DC in the mammal, thereby enhancing immunity in the mammal. The present invention further provides a method of inducing an immune response to an antigen, cancer, or infectious disease in a mammal. In one embodiment, the method comprises administering the antigen or an antigen of the cancer or infectious disease to a mammal, which has been treated as described above, whereupon an immune response to the antigen, cancer, or infectious disease, respectively, is induced in the mammal. In another embodiment, the method comprises administering a DC to a mammal as described above; however, the method further comprises contacting the DC, which has been modified to produce a dendritic cell-mediator, with the antigen or an antigen of the cancer or infectious disease prior to administration of the DC to the mammal, whereupon an immune response to the antigen, cancer or infectious disease, respectively, is induced in the mammal.

### CROSS-REFERENCE TO RELATED PATENT APPLICATIONS

This patent application is a continuation of copending U.S. patent application Ser. No. 09/586,702, filed Jun. 1, 2000, which claims the benefit of U.S. Provisional Patent Application No. 60/137,042, filed Jun. 1, 1999.

### STATEMENT REGARDING FEDERALLY SPONSORED RESEARCH AND DEVELOPMENT

This invention was made, in part, with funding from the United States Department of Health & Human Services, National Institutes of Health Grant No. R01 CA75192.

### FIELD OF THE INVENTION

The present invention relates to a method of enhancing immunity in a mammal by modifying a dendritic cell of the mammal to produce a dendritic cell-mediator, as well as a related method of inducing an immune response in a mammal.

### BACKGROUND OF THE INVENTION

Although many tumors have antigens recognizable by the immune system, the ability of tumors to escape a functional immune system suggests that the immune mechanism is often insufficient to overtake effectively the potential of many tumors to grow. An integral component of an antigen-specific immune response are dendritic cells. Dendritic cells (DC), which are potent antigen-presenting cells that function as the principal activators of quiescent T cells to initiate immune responses, reside in bone marrow, blood, organs frequently exposed to antigens, and lymphoid tissues. After interacting with antigens, immature DC undergo a maturation process as the cells migrate to lymphoid tissue, where the mature DC prime naive T cells.

Stimuli from CD4⁺ T-helper cells via the CD40/CD40 ligand (CD40L) interaction is essential in bringing the DC to a state in which they can autonomously trigger antigen-specific T-cell responses. CD40L is a 33 kDa, type-II membrane protein that is preferentially expressed on activated CD4⁺ T cells. The receptor for CD40L, CD40 (a 40 kDa protein), is expressed on antigen-presenting cells (APC), including DC, B cells and activated macrophages. In this context, the CD40L on antigen-stimulated CD4⁺ T-helper cells activates DC, with upregulation of T-cell costimulatory molecules, such as B7 and intercellular adhesion molecule-1 (ICAM-1), and consequently directs stimulation of CD8⁺ T-killer cells. As part of the activation of DC, the CD40/CD40L interaction induces the production of cytokines that favor the development of a Th1, or T cell-specific, response. However, whereas a role for DC in humoral immune responses is well established in the context of DC-mediated activation of CD4+ helper T cells, it is generally believed that DC cannot directly stimulate B cells to produce antigen-specific antibodies. Non-specific activation of B cells by DC has been observed when DC are activated.

Because tumor-specific immunity relies on professional APC activated through CD40/CD40L interactions, genetic modification of tumor cells, lymphoma cells, or bystander fibroblasts to express CD40L has been evaluated in an attempt to stimulate APC to induce tumor-specific cellular immunity. Adenovirus vectors have been used to transfer CD40L cDNA to human lymphoma cells and murine tumor cells; such transfer has induced generation of specific cytotoxic T lymphocytes (CTL). Murine tumor cells have been transduced ex vivo with a retroviral construct containing the CD40L cDNA, and a systemic immune response capable of impeding tumor growth in vivo has been induced. Triggering CD40 on murine lymphoblastic cells with retrovirus-mediated expression of CD40L on bystander fibroblasts has been shown to enhance the antigen-presenting potential of the lymphoblastic cells.

Finally, the CD40L-expressing plasmid has been transfected into murine mastocytoma cells, and its potentiation of host APC functions has been investigated. Based on this concept, others have shown that intradermal or subcutaneous co-injection of a plasmid expressing CD40L with a plasmid expressing β-galactosidase DNA enhances cellular immune response to β-galactosidase. Culture of DC with recombinant, soluble CD40L protein, fibroblasts or hybridoma cells transfected with CD40L cDNA, or anti-CD40 antibody, has induced APC functions of DC. This is associated with upregulation of accessory molecules, such as ICAM-1, B7-1 and B7-2, on CD40-triggered DC, and high levels of production of cytokines, such as IL-12, MIP-1α, IL-8 and TNF-α. Several studies have clearly illustrated that anti-CD40 antibody (acting as a surrogate of CD40L-triggered CD40 on DC) brings the DC to a state in which they can autonomously present antigen to CD8⁺ killer T cells and induce antigen-specific CTL responses. It has been recently shown that DC incubated with soluble CD40L protein mature to promote antitumor immunity in vivo, as evidenced by morphology, upregulation of adhesion and costimulatory molecules, and high levels of IL-12 secretion.

Unlike methods currently in existence, the present invention seeks to provide a method of self-activation of DC, which has several advantages. First, the self-activated DC can directly interact with various antigens in vivo without the possible alteration or concern for alteration or loss of antigen-associated RNA or peptides induced in in vitro manipulation. Second, in vivo interaction between the self-activated DC and cells expressing the entire repertoire of antigens should allow the host defense system to be stimulated against multiple cellular antigens. Third, administration of the self-activated DC does not depend on the prior identification of appropriate antigens and is not limited to expression of a particular corresponding MHC allele. For example, although it is possible to use some defined tumor antigens in DC adoptive transfer therapy, potential MHC-binding tumor-specific peptides remain unknown for most human tumors. Therefore, there is always the risk that ex vivo and in vitro enhancement of the immune response is not directed at the entire repertoire of antigens present in vivo.

In view of the above, the present invention seeks to provide a more effective method of enhancing immunity in a mammal. The present invention also seeks to provide a method of inducing an immune response in a mammal. These and other objects of the present invention, as well as additional inventive features and advantages, will be apparent from the description of the invention provided herein.

### BRIEF SUMMARY OF THE INVENTION

The present invention provides a method of enhancing immunity in a mammal. In one embodiment, the method comprises modifying a dendritic cell (DC) in the mammal to produce a dendritic cell-mediator. The dendritic cell-mediator up-regulates DC in the mammal, thereby enhancing immunity in the mammal.

In another embodiment, the method comprises removing a DC from the mammal, modifying the DC to produce a dendritic cell-mediator, and administering the modified DC to the mammal, whereupon the dendritic cell-mediator up-regulates DC, thereby enhancing immunity in the mammal. In yet another embodiment, the method comprises administering to the mammal a DC, which has been modified to produce a dendritic cell-mediator, whereupon the dendritic cell-mediator up-regulates DC, thereby enhancing immunity in the mammal.

The present invention further provides a method of inducing an immune response to an antigen in a mammal. In one embodiment, the method comprises administering the antigen to a mammal, which has been treated as described above, whereupon an immune response to the antigen is induced in the mammal. In another embodiment, the method comprises administering an antigen of a cancer to a mammal, which has cancer and has been treated as described above, whereupon an immune response to the cancer is induced in the mammal. In still yet another embodiment, the method comprises administering an antigen of an infectious disease to a mammal, which has an infectious disease and has been treated as described above, whereupon an immune response to a causative agent of the infectious disease is induced in the mammal. In yet another embodiment, the method comprises administering a DC to a mammal as described above; however, the method further comprises contacting the DC, which has been modified to produce a dendritic cell-mediator, with the antigen prior to administration of the DC to the mammal, whereupon an immune response to the antigen is induced in the mammal. In still yet another embodiment, the method comprises administering a DC to a mammal as described above; however, the mammal has cancer and the method further comprises contacting the DC, which has been removed from the mammal and modified to produce a dendritic cell-mediator, with an antigen of the cancer prior to administration of the DC to the mammal, whereupon an immune response to the cancer is induced in the mammal. In another embodiment, the method comprises administering a DC to a mammal as described above; however, the mammal has an infectious disease and the method further comprises contacting the DC, which has been modified to produce a dendritic cell-mediator, with an antigen of a causative agent of the infectious disease prior to administration of the DC to the mammal, whereupon an immune response to the infectious disease is induced in the mammal.

### BRIEF DESCRIPTION OF THE DRAWINGS

FIG. 1A is a bar graph of the cytokine IL-12 p40 (pg/ml) secreted by dendritic cells (DC) in which AdmCD40L, AdNull or PBS was administered.

FIG. 1B is a bar graph of the cytokine MIP-1α (pg/ml) secreted by DC in which AdmCD40L, AdNull or PBS was administered.

FIG. 2A is a graph of the specific ⁵¹Cr-release (%) of CT26-derived tumors injected with DC modified with AdmCD40L (□), AdNull (◯) or nothing (Δ) as a function of Effector/Target ratio.

FIG. 2B is a graph of the specific ⁵¹Cr-release (%) of B16-derived tumors injected with DC modified with AdmCD40L (□), AdNull (◯) or nothing (Δ) as a function of Effector/Target ratio.

FIG. 2C is a graph of the specific ⁵¹Cr-release (%) of C3-derived tumors injected with DC modified with AdmCD40L (□), AdNull (◯) or nothing (Δ) as a function of Effector/Target ratio.

FIG. 3A is a graph of survival (%) as a function of time (wks) when mice treated with splenocytes isolated from CT26-derived tumor-bearing mice previously treated with DC modified with AdmCD40L (□), AdNull (◯) or nothing (Δ) were exposed to the same tumor cells.

FIG. 3B is a graph of survival (%) as a function of time (wks) when mice treated with splenocytes isolated from B16-derived-tumor-bearing mice previously treated with DC modified with AdmCD40L (□), AdNull (◯) or nothing (Δ) were exposed to the same tumor cells.

FIG. 4A is a graph of tumor area (mm²) as a function of time (days) of mice bearing B16-derived flank tumors when untreated (□), or treated by intratumoral injection of DC modified with AdmCD40L (┘), AdNull (◯) or nothing (Δ).

FIG. 4B is a graph of survival (%) as a function of time (wks) of mice bearing B16-derived flank tumors when untreated (□), or treated by intratumoral injection of DC modified with AdmCD40L (▪), AdNull (◯) or nothing (Δ).

FIG. 5A is a graph of tumor area (mm³) as a function of time (days) of CT26-derived tumor-bearing Balb/c mice (H-2^{d}) untreated (□), or treated with 2×10⁶ DC modified with AdmCD40L (▪), AdNull (◯), or nothing (Δ).

FIG. 5B is a graph of survival (%) as a function of time (weeks) of CT26-derived tumor-bearing Balb/c mice (H-2^{d}) untreated (□), or treated with 2×10⁶ DC modified with AdmCD40L (▪), AdNull (◯), or nothing (Δ).

FIG. 5C is a graph of tumor area (mm³) as a function of time (days) of B16-derived tumor-bearing C57B1/6 mice untreated (□), or treated with 2×10⁶ DC modified with AdmCD40L (▪), AdNull (◯), or nothing (Δ).

FIG. 5D is a graph of survival (%) as a function of time (weeks) of B16-derived tumor-bearing C57B1/6 mice untreated (□), or treated with 2×10⁶ DC modified with AdmCD40L (▪), AdNull (◯), or nothing (Δ).

FIG. 6A is a graph of tumor area (mm³) as a function of time (days) of CT26-derived tumor-bearing Balb/c mice (H-2^{d}) untreated (□), or treated with 2×10⁵ DC modified with AdmCD40L (▪), AdNull (◯), or nothing (Δ).

FIG. 6B is a graph of survival (%) as a function of time (weeks) of CT26-derived tumor-bearing Balb/c mice (H-2^{d}) untreated (□), or treated with 2×10⁵ DC modified with AdmCD40L (▪), AdNull (◯), or nothing (Δ).

FIG. 6C is a graph of tumor area (mm³) as a function of time (days) of B16-derived tumor-bearing C57B1/6 mice untreated (□), or treated with 2×10⁵ DC modified with AdmCD40L (▪), AdNull (◯), or nothing (Δ).

FIG. 6D is a graph of survival (%) as a function of time (weeks) of B16-derived tumor-bearing C57B1/6 mice untreated (□), or treated with 2×10⁵ DC modified with AdmCD40L (▪), AdNull (◯), or nothing (Δ).

FIG. 7A is a graph of tumor area (mm²) as a function of time (days) of Balb/c mice treated with DC modified with AdmCD40L (□) or syngenic fibroblast CL7 cells modified with AdmCD40L (◯) and untreated (Δ).

FIG. 7B is a graph of specific ⁵¹Cr-release (%) as a function of Effector/Target ratio of Balb/c mice treated with DC modified with AdmCD40L (□) or syngenic fibroblast CL7 cells modified with AdmCD40L (◯) and untreated (Δ).

FIG. 8 is a graph of the number of migrated cells (per 5 high-powered fields (hpf)) as a function of the % supernatant as assayed by a modification of Boyden's chamber method using microchemotaxis chambers and filters (5 μm diameter) of the A549 lung carcinoma cell line infected with AdMIP-3α (▴), AdNull (□) or nothing (◯).

FIG. 9 is a graph of the number of migrated cells (per hpf) as a function of the % supernatant as assayed by a modification of Boyden's chamber method using microchemotaxis chambers and filters (5 μm diameter) of the A549 lung carcinoma cell line infected with AdSDF-1α (▴), AdNull (□) or nothing (◯).

FIG. 10A is a graph of tumor size (mm²) as a function of time (days) of the effects of AdMIP-3α administration on progression of pre-existing subcutaneous CT26.CL25-derived tumors in Balb/c mice after administration of AdNull (□), AdMIP-3α (▴), or nothing (◯).

FIG. 10B is a graph of tumor size (mm²) as a function of time (days) of the effects of AdMIP-3α administration on progression of pre-existing subcutaneous CT26-derived tumors in Balb/c mice after administration of AdNull (□), AdMIP-3α (▴), or nothing (◯).

FIG. 10C is a graph of tumor size (mm²) as a function of time (days) of the effects of AdMIP-3α administration on progression of pre-existing subcutaneous B16-derived tumors in C57B1/6 mice (H-2^{b}) after administration of AdNull (□), AdMIP-3α (▴), or nothing (◯).

FIG. 10D is a graph of tumor size (mm²) as a function of time (days) of the effects of AdMIP-3α administration on progression of pre-existing subcutaneous immunogenic Lewis lung carcinoma in C57B1/6 mice after administration of AdNull (□), AdMIP-3α (▴), or nothing (◯).

FIG. 10E is a graph of tumor size (mm²) as a function of time (days) of the effects of AdSDF-1α administration on progression of pre-existing subcutaneous CT-26-derived tumors in C57B1/6 mice after administration of AdNull (□), AdSDF-1α (▴), or nothing (◯).

FIG. 10F is a graph of tumor size (mm²) as a function of time (days) of the effects of AdSDF-1α administration on progression of pre-existing subcutaneous B16-derived tumors in Balb/c mice after administration of AdNull (□), AdSDF-1α (▴) or nothing (◯).

FIG. 10G is a graph of tumor size (mm²) as a function of time (days) of the effects of AdSDF-1α administration on progression of pre-existing subcutaneous immunogenic Lewis lung carcinoma in C57B1/6 mice (H-2^{b}) after administration of AdNull (□), AdSDF-1α (▴) or nothing (◯).

FIG. 10H is a graph of survival (%) as a function of time (wks) of the effects of AdMIP-3α administration on progression of pre-existing subcutaneous CT26.CL25-derived tumors in Balb/c mice after administration of AdNull (□), AdMIP-3α (▴) or nothing (◯).

FIG. 10I is a graph of survival (%) as a function of time (wks) of the effects of AdMIP-3α administration on progression of pre-existing subcutaneous CT26-derived tumors in Balb/c mice after administration of AdNull (□), AdMIP-3α (▴) or nothing (◯).

FIG. 10J is a graph of survival (%) as a function of time (wks) of the effects of AdMIP-3α administration on progression of pre-existing subcutaneous B16-derived tumors in C57B1/6 mice (H-2^{b}) after administration of AdNull (□), AdMIP-3α (▴) or nothing (◯).

FIG. 10K is a graph of survival (%) as a function of time (wks) of the effects of AdMIP-3α administration on progression of pre-existing subcutaneous immunogenic Lewis lung carcinoma in C57B1/6 mice after administration of AdNull (□), AdMIP-3α (▴) or nothing (◯).

FIG. 11A is a graph of percent lysis as a function of Effector/Target ratio of cytotoxic T cells following intratumoral administration of AdMIP-3α to CT26.CL25-derived tumors in Balb/c mice after administration of AdNull (□), AdMIP-3α (▴) or nothing (◯).

FIG. 11B is a graph of percent lysis as a function of Effector/Target ratio of cytotoxic T cells following intratumoral administration of AdMIP-3α to CT26-derived tumors in Balb/c mice after administration of AdNull (□), AdMIP-3α (▴) or nothing (◯).

FIG. 11C is a graph of percent lysis as a function of Effector/Target ratio of cytotoxic T cells following intratumoral administration of AdMIP-3α B16-derived tumors in Balb/c mice after administration of AdNull (□), AdMIP-3α (▴) or nothing (◯).

FIG. 11D is a graph of percent lysis as a function of Effector/Target ratio of cytotoxic T cells following intratumoral administration of AdMIP-3α to Lewis lung carcinoma in Balb/c mice after administration of AdNull (□), AdMIP-3α (▴) or nothing (◯).

FIG. 12A is a graph of survival (%) as a function of time (wks) of the ability of adoptive transfer of splenocytes from syngenic mice treated with AdMIP-3α to protect recipient mice from growth of subcutaneous CT26.CL25-derived tumors after administration of AdMIP-3α-modified splenocytes (▴), AdNull-modified splenocytes (□) or untreated splenocytes (◯)or nothing (●).

FIG. 12B is a graph of survival (%) as a function of time (wks) of the ability of adoptive transfer of splenocytes from syngenic mice treated with AdMIP-3α to protect recipient mice from growth of subcutaneous CT26-derived tumors after administration of AdMIP-3α-modified splenocytes (▴), AdNull-modified splenocytes (□), untreated splenocytes (◯), or nothing (●).

FIG. 12C is a graph of survival (%) as a function of time (wks) of the ability of adoptive transfer of splenocytes from syngenic mice treated with AdMIP-3α to protect recipient mice from growth of subcutaneous B16-derived tumors after administration of AdMIP-3α-modified splenocytes (▴), AdNull-modified splenocytes (□), untreated splenocytes (◯), or nothing (●).

FIG. 12D is a graph of survival (%) as a function of time (wks) of the ability of adoptive transfer of splenocytes from syngenic mice treated with AdMIP-3α to protect recipient mice from growth of subcutaneous Lewis lung carcinoma-derived tumor after administration of AdMIP-3α-modified splenocytes (▴), AdNull-modified splenocytes (□), untreated splenocytes (◯), or nothing (●).

### DETAILED DESCRIPTION OF THE INVENTION

The present invention is predicated on the unexpected benefits of self-activation of dendritic cells (DC). The benefits of self-activation include the ability of self-activated DC to directly interact with various antigens without the possible alteration or loss of antigen-associated RNA or peptides induced in antigen-related manipulations. Also, in vivo interaction between the self-activated DC and an entire repertoire of antigens allows the host defense system to be stimulated against multiple cellular antigens. Furthermore, administration of the self-activated DC does not depend on the prior identification of appropriate antigens and is not limited to expression of a particular corresponding MHC allele. For example, potential MHC-binding tumor-specific peptides are unknown for most human tumors and without a precise knowledge of all of the tumor antigens, the immune response is not directed at the entire repertoire of antigens present.

The invention may best be understood with reference to the following detailed description of the preferred embodiments. The present invention provides a method of enhancing immunity in a mammal. In one embodiment, the method comprises modifying a DC in the mammal to produce a dendritic cell-mediator, whereupon the dendritic cell-mediator up-regulates DC, thereby enhancing immunity in the mammal. In another embodiment, the method comprises removing a DC from the mammal, modifying the DC to produce a dendritic cell-mediator, and administering the modified cell to the mammal, whereupon the dendritic cell-mediator up-regulates DC, thereby enhancing immunity in the mammal. In still yet another embodiment, the method comprises administering to the mammal a DC, which has been modified to produce a dendritic cell-mediator, whereupon the dendritic cell-mediator up-regulates DC, thereby enhancing immunity in the mammal.

Any suitable dendritic cell-mediator can be used in the context of the present invention. By “dendritic cell-mediator” is meant any molecule that up-regulates DC as described herein, such as by directly increasing DC maturation or migration. Examples of DC-mediators include lipopolysaccharide (LPS), granulocyte-macrophage colony stimulating factor (GM-CSF), interleukin-1β (IL-1β), tumor necrosis factor-α (TNFα), Flt-3 ligand, and c-kit. In addition to C5a, platelet activating factor (PAF) and formyl peptides (FMLP), several chemokines induce directional migration of Langerhans' cells, monocyte-derived DC and CD34+ cell-derived DC in vitro, including the CC chemokines RANTES (regulated on activation, normal T cell expressed and secreted), macrophage inflammatory protein (MIP)-1α, MIP-1β, MIP-3α, monocyte chemotactic protein (MCP)-3, MCP-4, MIP-5/human CC cytokine-2 (HCC2), macrophage-derived chemokine (MDC) and stromal cell-derived factor-1α (SDF-1α). Preferably, the dendritic cell-mediator increases DC maturation. Any suitable dendritic cell-mediator that increases DC maturation can be used in the context of the present inventive methods. Examples of DC-mediators that increase DC maturation include CD40L, TNF, interleukin-3 (IL-3), and GM-CSF. A preferred dendritic cell-mediator that increases DC maturation is CD40 ligand (CD40L), which binds to CD40 on the surfaces of the DC.

The term “CD40L” is defined as any protein (or polypeptide or peptide) that binds CD40, which is present on the surface of DC, including those proteins that are well-known in the art, see, generally, Lederman et al., Curr. Opin. Hematol., 3: 77-86 (1996); Grewal and Flavell, Immunol. Today, 17: 410-14 (1996); Buhlmann and Noelle, J. Clin. Immunol., 16: 83-89 (1996); Laman et al., Crit. Rev. Immunol., 16: 59-108 (1996); Noelle, Clin. Immunol. Immunopathol., 76: S203-7 (1995); Castigli et al., Int. Arch. Allerg. Immunol., 107: 37-39 (1995); Ochs et al., Semin. Immunol., 6: 337-41 (1994); Fanslow et al., Semin. Immunol., 6: 267-78 (1994); Armitage et al., Semin. Immunol., 5: 401-12 (1993). In addition, in the context of the present invention, CD40L includes those molecules that have been altered through addition, subtraction, or substitution, either conservatively or non-conservatively, of any number of amino acids, provided that the resulting protein binds CD40 on the surface of DC. A “conservative alteration” is one in that results in an alternative amino acid of similar charge density, hydrophilicity or hydrophobicity, size, and/or configuration (e.g., Val for Ile). In comparison, a “nonconservative alteration” is one that results in an alternative amino acid of differing charge density, hydrophilicity or hydrophobicity, size, and/or configuration (e.g., Val for Phe). The means of making such modifications are well-known in the art and also can be accomplished by means of commercially available kits and vectors (for example, those available from New England Biolabs, Inc., Beverly, Mass.; Clontech, Palo Alto, Calif.).

Alternatively and also preferably, the dendritic cell-mediator increases DC migration. Any suitable dendritic cell-mediator that increases DC migration can be used in the context of the present inventive methods. Examples of DC-mediators that increase DC migration include MIP-3α, MIP-3β, IL-1, SDF-1α and other chemokines, lymphokines, etc. Preferably, the DC-mediator is a chemokine, more preferably, the chemokine is MIP-3α or SDF-1α. MIP-3α is a 8.0 kDa CC chemokine that, in addition to being chemotactic for DC in vitro, is chemotactic for lymphocytes, but not for monocytes or neutrophils. Immature DC derived from CD34+ hematopoietic progenitor cells migrate vigorously in response to MIP-3α, but upon maturation, the DC lose their response to this chemokine. Consistent with this observation, mRNA levels for CCR6 (the MIP-3α receptor) are high in immature DC, and decrease as DC mature. In normal mammals, the MIP-3α gene is expressed in lung, appendix, liver, and some lymphoid tissues, as well as in activated endothelial cells and monocytes. Indirect evidence that MIP-3α might attract DC in vivo comes from studies showing MIP-3α mRNA within inflamed epithelial crypts of tonsils. SDF-1α is a CXC chemokine and is a potent chemo-attractant for DC, likely playing a role in the directional migration of DC in vivo, see, generally, Broxmeyer et al., Ann. N. Y. Acad. Sci. 872:142-62 (1999). SDF-1α has also been shown to induce accumulation and infiltration of CD8+T cells in tumors.

Other factors increase stimulation of T-cells and B-cells. For example, IL-2, IL-12 and the like stimulate T-cells. IL-4 and the like also stimulate B-cells.

The DC can be modified in the context of the present inventive methods by any suitable means. Preferably, the DC is modified in the context of the present inventive methods by contacting the DC with a nucleic acid molecule comprising a nucleic acid sequence encoding a dendritic cell-mediator as described above. Preferably, the nucleic acid molecule is a viral vector. Preferably, the viral vector is an adenoviral vector. Preferably, the adenoviral vector is deficient in at least one essential gene function of the E1 region of the adenoviral genome and, optionally, has a deficiency in the E3 region, and/or the adenoviral vector is deficient in at least one essential gene function of the E4 region of the adenoviral genome. Preferably, the nucleic acid sequence encoding a dendritic cell-mediator is positioned in the E1 region of the adenoviral genome.

While any suitable means of contacting can be used within the context of the present invention, preferably, such contacting is accomplished by directly injecting the nucleic acid molecule comprising a nucleic acid sequence encoding a DC-mediator into the mammal, by catheter or like device, or by topically applying the nucleic acid molecule comprising a nucleic acid sequence encoding a DC-mediator to the mammal. By the term “injecting,” it is meant that the nucleic acid molecule comprising a nucleic acid sequence encoding a DC-mediator is forcefully introduced into the mammal. Any suitable injection device can be used within the context of the present invention. Furthermore, the DC-mediator can be administered subcutaneously, into airways, orally, intravenously, intra-muscularly, or otherwise. Preferably, in embodiments where the mammal has a tumor or infectious disease, the DCs that are modified with the nucleic acid molecule comprising a nucleic acid sequence encoding a DC-mediator are in the vicinity of the tumor or infectious disease.

Furthermore, the DC-mediator can be specifically administered to DC. This can be accomplished by any suitable method, such as, for example, the DC-mediator can be administered in combination with an agent that directs binding of the DC-mediator to the DC. When a nucleic acid molecule encodes the DC-mediator, the nucleic acid sequence encoding the DC-mediator can be operably liked to a regulatable element, such as a tissue-specific promoter. Furthermore, when a viral vector encodes the DC-mediator, the viral vector can include a targeting agent that directs binding of the vector to the DC. Such a targeting agent includes, for example, a bi-specific molecule or a chimeric viral coat protein, which are known in the art and described generally in U.S. Pat. No. 5,770,442. Other suitable modifications to the vector are described in U.S. Pat. Nos. 5,559,099, 5,731,190, 5,712,136, and 5,846,782 and International Patent Applications WO 97/20051, WO 98/07877, and WO 98/54346.

Alternatively, the DCs can be modified to produce a DC-mediator ex vivo. Removal of the DCs can be accomplished by any suitable method, for example, from bone marrow precursor cells harvested from a femur. See Inaba et al., J. Exp. Med., 176: 1693 (1992). After modification, the DCs are administered to the mammal, which can be accomplished by any suitable method, for example, direct injection. Preferably, in situations where the mammal has a tumor or infectious disease, the modified cells are administered to the mammal in the vicinity of the tumor or infectious disease.

If the mammal has a cancer, the above methods can be used to enhance an immune response to the cancer in the mammal. If the cancer is in the form of a tumor, desirably, the DC that is modified is in the vicinity of the tumor. Furthermore, if the mammal has been infected with a disease, the above methods can be used to enhance an immune response to the infectious disease.

In view of the above, the present invention further provides a method of inducing an immune response to an antigen in a mammal. In one embodiment, the method comprises administering to a mammal, which has been treated in accordance with an above-described method, the antigen, whereupon an immune response to the antigen is induced in the mammal. In another embodiment, the mammal has cancer or an infectious disease, and the method comprises administering to the mammal, which has been treated in accordance with an above-described method, an antigen of the cancer or infectious disease, whereupon an immune response to the cancer or infectious disease, respectively, is induced in the mammal. The antigen of the cancer can be a cell of the cancer. In yet another embodiment, the method comprises administering a DC to a mammal in accordance with an above-described method, which method further comprises contacting the DC, which has been removed from the mammal and modified to produce a dendritic cell-mediator, with the antigen prior to administration of the DC to the mammal, whereupon an immune response to the antigen is induced in the mammal. In still yet another embodiment, the mammal has cancer or an infectious disease and the method comprises administering a DC to the mammal in accordance with an above-described method, which method further comprises contacting the DC, which has been modified to produce a dendritic cell-mediator, with an antigen of the cancer or infectious disease prior to administration of the DC to the mammal, whereupon an immune response to the cancer or infectious disease, respectively, is induced in the mammal. The antigen of the cancer can be a cell of the cancer.

By up-regulation of DC, it is meant that the function of the DC is improved. For example, the improvement in the DC may be due to proliferation of the DC, maturation of the DC, migration of the DC, recruitment by the DC of other cells, expression of cytokine or chemokines, increased presentation of antigens by the DC, or any other modification that serves to improve the function or efficiency of the DC.

By “enhancing an immune response” is meant any improvement in an immune response that has already been mounted by a mammal. By “inducing an immune response” is meant the initiation of an immune response against an antigen of interest in a mammal in which an immune response against the antigen of interest has not already been initiated. In both situations, the immune response can involve both the humoral and cell-mediated arms of the immune system. For further discussion of immune responses, see, e.g., Abbas et al. Cellular and Molecular Immunology, 3^{rd} Ed., W. B. Saunders Co., Philadelphia, Pa. (1997).

The cell-mediated or local immune response is produced by T cells, which are able to detect the presence of invading pathogens through a recognition system referred to as the T cell antigen receptor. Upon detection of an antigen, T cells direct the release of multiple T cell cytokines, including IL-2, IL-3, IFN-γ, TNF-β, GM-CSF and high levels of TNF-α, and chemokines MIP-1α, MIP-1 β, and RANTES. IL-2 is a T cell growth factor that promotes the production of additional T cells sensitive to the particular antigen. This production constitutes a clone of the T cells. The sensitized T cells attach to cells containing the antigen. T cells carry out a variety of regulatory and defense functions and play a central role in immunologic responses. When stimulated to produce a cell-mediated immune response, some T cells respond by acting as killer cells, killing the host's own cells when these cells are infected or cancerous and therefore recognized as foreign. Some T cells respond by stimulating B cells, while other T cells respond by suppressing immune response. Therefore, if a cell-mediated immune response occurs, T cells are activated and cytokines, specifically IFN-γ, and chemokines are produced.

The humoral or systemic immune response depends on the ability of the B cells to recognize specific antigens. The mechanism by which B cells recognize antigens is through specific receptors on the surface of the B cells. When an antigen attaches to the receptor site of a B cell, the B cell is stimulated to divide. The daughter cells become plasma cells that manufacture antibodies complementary to the attached antigen. Each plasma cell produces thousands of antibody molecules per minute, which are released into the bloodstream. Many B cells appear to be regulated by the helper T cells and suppressor T cells and produce various cytokines, e.g., IL-3, IL-4, IL-5, IL-6, IL-9, IL-10, IL-13, GM-CSF and low levels of TNF-α. Helper T cells stimulate B cells to produce antibodies against antigens, while suppressor T cells inhibit antibody production. Some B cells, however, are T cell independent and require no stimulation by the T cells. Therefore, if a humoral immune response occurs, B cells are activated and cytokines, specifically IL-6, are produced.

Enhancement of an immune response can thus be determined by measuring the improvement in production by the mammal of the specific cytokines and chemokines for the two arms of the immune system. For example, to determine improvement in the humoral immune response, an increase in IL-6 can be determined, whereas to determine improvement in the cell-mediated immune response, an increase in IFN-γ can be determined.

Optionally, the nucleic acid molecule comprising a nucleic acid sequence encoding a DC-mediator can be contained within, conjugated with or co-administered with a nucleic acid molecule, protein, hydrocarbon and/or lipid. Examples of suitable nucleic acid molecules include fusion or chimeric nucleic acids, proteins, hydrocarbons and/or lipids. Co-administration can be such that the nucleic acid molecule comprising a nucleic acid sequence encoding a DC-mediator is administered before, at substantially the same time as, or after the other nucleic acid, protein, hydrocarbon, and/or lipid. Preferably, the nucleic acid molecule comprising a nucleic acid sequence encoding a DC-mediator is administered at substantially the same time as the other nucleic acid, protein, hydrocarbon, and/or lipid. The present invention also can be combined with any other method currently known and used in the art requiring enhancement or induction of an immune response; for example, methods of suppressing tumor growth in a mammal, which include surgical removal of the cancerous tissue, radiation therapy, chemotherapy, and administration of immune-response inducing compositions or vaccines against viral, bacterial or parasitic infectious organisms.

A number of methods are available to deliver the nucleic acid molecule comprising a nucleic acid sequence encoding a DC-mediator, including particle bombardment, transfection and transduction. The nucleic acid molecule comprising a nucleic acid sequence encoding a DC-mediator can be, for example, plasmid nucleic acid molecule comprising a nucleic acid sequence, plasmid-liposome complexes, or a viral vector such as adenovirus, herpes simplex virus (HSV), or adeno-associated virus (AAV).

Plasmids, genetically engineered circular double-stranded DNA molecules, can be designed to contain an expression cassette for delivery of a specific DNA. Although plasmids were the first method described for gene transfer of DNA, their level of efficiency is poor, compared with other techniques. By complexing the plasmid with liposomes, the efficiency of gene transfer in general is improved. While the liposomes used for plasmid-mediated gene transfer strategies have various compositions, they are typically synthetic cationic lipids. The positively charged liposome forms a complex with a negatively charged plasmid. These plasmid-liposome complexes enter target cells by fusing with the plasma membrane. Advantages of plasmid-liposome complexes include their ability to transfer large pieces of exogenous DNA and their relatively low potential to evoke immunogenic responses in the host.

The adenovirus is a 36 kb double-stranded DNA virus that efficiently transfers DNA in vivo to a variety of different target cell types, including skeletal muscle. The virus is made suitable by deleting some of the genes required for viral replication; the expendable E3 region is also frequently deleted to allow additional room for a larger DNA insert. The resulting replication-deficient adenoviral vector can accommodate up to 7.5 kb of exogenous DNA. The vector can be produced in high titers and is capable of efficiently transferring DNA to replicating and non-replicating cells. This is of particular importance for transfer of DNA to the skeletal muscle, in which the host cell is terminally differentiated. The newly transferred genetic information remains epi-chromosomal, thus eliminating the risks of random insertional mutagenesis and permanent alteration of the genotype of the target cell.

HSV is another viral vector that has been used to accomplish administration of exogenous DNA. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the herpes vector are its ability to enter a latent stage that could potentially result in long-term DNA expression, and its large viral DNA genome that can accommodate exogenous DNA up to 25 kb.

AAV vectors represent another potential approach to administering exogenous DNA. AAV is a DNA virus that is not known to cause human disease and that requires coinfection by a helper virus (i.e., adenovirus or herpes virus) for efficient replication. AAV vectors used for administration of exogenous DNA have approximately 96% of the parental genome deleted such that only the terminal repeats remain, which contain recognition signals for DNA replication and packaging. This eliminates immunologic or toxic side effects due to expression of viral genes.

Preferably, the nucleic acid molecule comprising a nucleic acid sequence encoding either a DC-mediator is an adenoviral vector. The adenoviral vector is preferably deficient in at least one gene function required for viral replication. Preferably, the adenoviral vector is deficient in at least one essential gene function of the E1 region of the adenoviral genome, particularly the E1a region, more preferably, the vector is deficient in at least one essential gene function of the E1 region and part of the E3 region (e.g., an Xba I deletion of the E3 region) or, alternatively, the vector is deficient in at least one essential gene function of the E1 region and at least one essential gene function of the E4 region. However, adenoviral vectors deficient in at least one essential gene function of the E2a region and adenoviral vectors deficient in all of the E3 region also are contemplated here and are well-known in the art. Adenoviral vectors deleted of the entire E4 region can elicit lower host immune responses. Suitable replication-deficient adenoviral vectors are disclosed in International Patent Applications WO 95/34671 and WO 97/21826. For example, suitable replication-deficient adenoviral vectors include those with a partial deletion of the E1a region, a partial deletion of the E1b region, a partial deletion of the E2a region, and a partial deletion of the E3 region. Alternatively, the replication-deficient adenoviral vector can have a deletion of the E1 region, a partial deletion of the E3 region, and a partial deletion of the E4 region.

Furthermore, the adenoviral vector's coat protein can be modified so as to decrease the adenoviral vector's ability to be recognized by a neutralizing antibody directed against the wild-type coat protein, as described in International Patent Application WO 98/40509. Other suitable modifications to the adenoviral vector are described in U.S. Pat. Nos. 5,559,099 (Wickham et al.), 5,731,190 (Wickham et al.), 5,712,136 (Wickham et al.), and 5,846,782 (Wickham et al.) and International Patent Applications WO 97/20051, WO 98/07877, and WO 98/54346.

The nucleic acid molecule comprising a nucleic acid sequence, operably linked to expression signals and encoding either a DC-mediator, can be inserted into any suitable region of the adenoviral vector as an expression cassette. In that respect, the ordinarily skilled artisan will readily appreciate that there are certain advantages to using an adenoviral vector deficient in some essential gene region of the adenoviral genome inasmuch as such a deficiency will provide room in the vector for a transgene and will prevent the virus from replicating. Preferably, the nucleic acid molecule comprising a nucleic acid sequence is inserted into the E1 region of the adenoviral vector. Whereas the nucleic acid molecule comprising a nucleic acid sequence can be inserted as an expression cassette in any suitable orientation in any suitable region of the adenoviral vector, preferably, the orientation of the DNA segment is from right to left. By the expression cassette having an orientation from right to left, it is meant that the direction of transcription of the expression cassette is opposite that of the region of the adenoviral vector into which the expression cassette is inserted.

In the context of the present inventive methods, any suitable antigen can be administered to a mammal or contacted with a dendritic cell. The antigen can be any suitable molecule recognized by the immune system of the mammal as foreign. For example, the antigen can be any foreign molecule, such as a protein (including a modified protein such as a glycoprotein, a mucoprotein, etc.), a nucleic acid, a carbohydrate, a proteoglycan, a lipid, a mucin molecule, or other similar molecule, including any combination thereof. The antigen can also be a cell or a part thereof, for example, a cell surface molecule. Further, a suitable antigen can be an infectious virus, bacteria, fungi, and other organism (e.g., protists), or part thereof. These infectious organisms can be active or inactive, which can be accomplished, for example, through exposure to heat or removal of at least one protein or gene required for replication of the organism. Preferably, the antigen administered to the mammal or contacted with the DC includes more than one antigen, such that the immune response generated is directed to a large repertoire, preferably, the entire repertoire of antigens.

In the context where the mammal has a cancer, the antigen is a cancer antigen, which includes cells of the cancer, cell surface molecules, or any other molecule present in a cancer cell. As discussed previously, the cancer antigen can include more than one antigen. Furthermore, when the mammal has an infectious disease, the antigen is an antigen from the infectious organism causing the infectious disease, which includes cells of the organism, cell surface molecules, or any other molecule present in the organism (as discussed previously). Again, the antigen can include more than one infectious disease antigen.

Any suitable method of administering the antigen to a mammal can be used in the present inventive methods. For example, the antigen can be administered to the mammal by directly injecting the antigen into the mammal, by catheter or like device, or by topically applying the antigen to the mammal (as discussed previously). Alternatively, the antigen can be contacted with the DC ex vivo. In an ex vivo context, the antigen can be contacted with the DC by any suitable method. One such method is by direct administration of the antigen to the culture medium in which the DC is maintained.

Furthermore, the antigen to be administered to the mammal or contacted with the DC can encoded by a nucleic acid molecule. Suitable nucleic acid molecules include, for example, plasmids, plasmid-liposome complexes, or viral vectors, such as adenovirus, herpes simplex virus (HSV), or adeno-associated virus (AAV) vectors (which have been described herein). Preferably, if the antigen is encoded by a nucleic acid molecule, it is encoded by a viral vector, and more preferaby, an adenoviral vector (which have be described herein).

The nucleic acid molecule comprising a nucleic acid sequence encoding a DC-mediator can also encode the antigen. It is preferable that an adenoviral vector be utilized to co-administer the antigen and DC-mediator. Co-administration of the antigen and DC-mediator has many advantages. For example, co-administration ensures that the antigen and DC-mediator are present in the mammal at the same time and in the same general location, thereby increasing the antigen specificity of the immune response generated.

While not wishing to be bound by any particular theory, it is generally thought that DC cannot stimulate cytotoxic T-cells directly unless they are first stimulated via CD40 on their surface, which is usually accomplished by CD40L expressed on CD4⁺ helper T cells. The T-helper-mediated CD40 triggering up-regulates adhesion and co-stimulatory molecules in the DC, bringing the DC to a state where they can autonomously stimulate a T-killer response. The expression of CD40L is normally restricted almost exclusively to activated CD4⁺ helper T cells, and is exquisitely regulated in concert with other receptor-ligand pairs within a specialized microenvironment.

Modification of DC to express CD40L, for example, accomplishes the goal of self-activating DC to induce functionally relevant cell-mediated adoptive immune responses, such as suppression of tumor growth in an antigen-specific fashion. It is thought that following administration of the DNA encoding CD40L to DC to produce CD40L, the transduced DC self-trigger CD40 and self-activate to present tumor antigen to naive CD8⁺ CTL. When administered to tumors, these genetically modified DC capture tumor antigens and present them to naive CD8⁺ CTL after migrating to lymphoid organs. DC expressing CD40L can also activate a humoral immune response, such that, upon introduction of an antigen, B cells can be triggered to produce antigen-specific antibodies without CD4⁺ T cell help.

The triggering of CD40 on DC leads to increased production of several inflammatory cytokines and chemokines, including interleukin-12 (IL-12) and MIP-1α. IL-12, a cytokine, which promotes the development of T helper-1 (Th1) CD4⁺ T cells and the maturation of CTL, likely plays a supportive role for generation of T-killer responses for tumor immunity. In contrast, MIP-1α, a chemokine known to induce preferentially the migration of CD8⁺ T cells, helps DC to encounter and stimulate rare tumor antigen-specific CD8⁺ CTL.

The present inventive method can be used to treat any suitable condition that involves an immune response and can benefit from an enhanced immune response. Examples of such conditions include, for example, cancer, immune system deficiencies or disorders and infectious diseases.

The present inventive method can be used to treat any suitable cancer alone or in combination with any suitable anti-cancer agent. Suitable cancers include cancers of the skin (e.g., melanoma), brain, lung (e.g., small cell and non-small cell), ovary, breast, prostate, and colon, as well as other carcinomas and sarcomas. Suitable anti-cancer agents include those substances given in treatment of the various conditions described above, examples of which include cytotoxic agents, such as alkylating agents and cisplatin. Other suitable anti-cancer agents can be found in the Physicians' Desk Reference (1998).

In one embodiment, the mammal has a cancer or a tumor and enhancement of immunity suppresses growth of the cancer or tumor in the mammal. By the term “suppresses cancer growth” or “suppression of cancer growth,” it is meant that growth of a cancer is halted or the rate of cancer growth is reduced. By the term “suppresses tumor growth” or “suppression of tumor growth,” it is meant that growth of a tumor is halted or the rate of tumor growth is reduced. Therefore, the present method provides for the size of a cancer or a tumor to be reduced, remain the same, or even increase, but at a decreased rate.

The present inventive method can be used to treat, prevent, or ameliorate any suitable disease associated with the immune system. Preferred diseases associated with the immune system are autoimmune disorders and immune system deficiencies, e.g., lupus erythematosus, and autoimmune diseases such as rheumatoid arthritis and multiple sclerosis. Immune system deficiencies include those diseases or disorders in which the immune system is not functioning at normal capacity, or in which it would be useful to boost the immune system response.

The present inventive method can be used to treat, prevent, or ameliorate any suitable infection alone or in combination with any suitable anti-infectious agent. Examples include francisella, schistosomiasis, tuberculosis, AIDS, malaria, and leishmania. Examples of suitable infectious viruses, bacteria, fungi, and other organisms (e.g., protists) can be found in International Patent Application WO 98/18810. Suitable anti-infectious agents include those substances given in treatment of the various conditions described elsewhere, examples of which can be found in the Physicians' Desk Reference (1998).

In one embodiment, the mammal has an infectious disease and enhancement of immunity suppresses infection of the disease in the mammal. By the term “suppresses infection” or “suppression of infection,” it is meant that the rate of infection is maintained or the rate of infection is reduced. Therefore, the present method provides for the rate of infection to be reduced, remain the same, or even increase, but at a decreased rate.

The present inventive method can also be used to improve the efficacy of any composition that induces an immune response or any suitable vaccine. Suitable vaccines include those directed against Hepatitis A, B, and C or any other suitable infection, examples of which can be found in the Physicians' Desk Reference (1998), and DNA vaccines directed against HIV and malaria. See generally Klinman et al., Vaccine 17: 19 (1999); McCluskie & Davis, J. Immun. 161: 4463 (1998).

One possible application of the present inventive method of vaccination is in the treatment of patients with cystic fibrosis (CF), a hereditary disorder caused by mutations in the cystic fibrosis transmembrane conductance regulator (CFTR) gene, characterized in most affected individuals as a chronic respiratory tract disease manifested early in life by progressive derangements of the airways and recurrent pulmonary infection. The lungs of CF patients are particularly susceptible to the gram-negative bacterium Pseudomonas aeruginosa, and chronic infection with this organism is strongly associated with the development and progression of pulmonary disease in these individuals. The typical patient with CF has repeated cycles of exacerbations of pulmonary infection with Pseudomonas, leading to an intense inflammatory response on the airway epithelial surface, deterioration of lung function, bronchiectasis, respiratory failure and eventual death. Although there have been significant improvements in the antibiotic therapy of Pseudomonas infection, Pseudomonas infection remains a major management problem in CF.

Among Pseudomonas antigens, several types of molecules have been evaluated for immunogenicity as a vaccine, including lipopolysaccharide (LPS), the mucoid exopolysaccharide (MEP; also called alginate) in the mucoid capsule surrounding bacteria, different portions of cell surface LPS (O-side-chain polysaccharide, core oligosaccharide, neutral polysaccharide and lipid A portions), outer membrane proteins (OPR), a polar protein filament (flagella), the exotoxin A (ETA), several proteases, hemolysins, anti-idiotypic antibody mimicking Pseudomonas LPS and whole cells (live, killed, extracts and sonicates). Sawa et al., Nat.Med. 5: 392 (1999) recently reported a novel strategy for developing a Pseudomonas vaccine, showing that PcrV (P. aeruginosa homolog of the Yersina V antigen) is involved in the translocation of toxins into eukaryotic cells, and that vaccination against PcrV ensured the survival of challenged mice with decreased lung injury.

The ability to modify DC genetically to express CD40L, for example, ex vivo and induce antigens specific to Pseudomonas by pulsing the DC with Pseudomonas has several theoretical advantages with potential clinical interest. First, pulsing DC with whole-cell Pseudomonas should allow the host immune system to be stimulated by multiple Pseudomonas antigens. The involvement of oligoclonal effectors specific for diverse antigenic epitopes will likely contribute to an optimal anti-Pseudomonas response.

In addition, the fact that CD4⁺ T cells are not required to induce protective immunity of transduced DC, such as CD40L-transduced DC, may support the usefulness of vaccination to enhance specific immunity even in immunocompromised patients, especially patients who are suffering from acquired immunodeficiency syndrome (AIDS) characterized by a defective function of CD4⁺ T cell help.

Desirably, administration to the mammal utilizes a pharmaceutical composition, which comprises a pharmaceutically acceptable carrier. Any suitable pharmaceutically acceptable carrier can be used within the context of the present invention, and such carriers are well-known in the art. The choice of carrier will be determined, in part, by the particular site to which the composition is to be administered and the particular method used to administer the composition. Formulations suitable for injection include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous injection solutions and suspensions can be prepared from sterile powders, granules, and tablets of the kind previously described. Preferably, the pharmaceutically acceptable carrier is a buffered saline solution.

Administration to a mammal of a DC-mediator, a modified dendritic cell that expresses a DC-mediator, alone or in further combination with an antigen, together or separately, or a modified dendritic cell that expresses a DC-mediator and that has been contacted with an antigen ex vivo should be such as to enhance an immune response or induce an immune response in the mammal as appropriate over a reasonable period of time. The amount of a DC-mediator, which preferably is encoded in a nucleic acid molecule, administered to the mammal should be sufficient to up-regulate DC in the mammal. If the DC-mediator is encoded in a nucleic acid molecule, the amount of nucleic acid molecule administered to the mammal should be sufficient to infect, transduce or transform a dendritic cell, desirably multiple dendritic cells, in the mammal so as to effect expression of the DC-mediator and the enhancement/induction of an immune response. The amount of a nucleic acid molecule encoding a DC-mediator to administer to a mammal to achieve a sufficient level of expression of the encoded DC-mediator can be determined in accordance with methods known in the art.

Generally, an amount of nucleic acid molecule comprising a nucleic acid encoding a DC-mediator sufficient to achieve a tissue concentration of about 10² to about 10¹² viral particles per ml is preferred, especially of about 10⁶ to about 10¹⁰ viral particles per ml. In certain applications, multiple daily doses are preferred. Moreover, the number of doses will vary depending on the means of delivery. For example, for an adenoviral vector comprising the nucleic acid molecule comprising a nucleic acid encoding a DC-mediator, a dose typically will be at least about 1×10⁶ to about 1×10¹³ particle forming units (pfu) to the mammal, regardless of the method by which the cells are modified. For purposes of considering the dose in terms of particle units (pu), also referred to as viral particles, it can be assumed that there are 100 particles/pfu (e.g., 1×10¹² pfu is equivalent to 1×10¹⁴ pu).

If a dendritic cell is removed from the mammal and modified to express a DC-mediator, desirably multiple dendritic cells are removed and modified as described herein above. The amount of modified dendritic cells administered to the mammal also should be sufficient to enhance an immune response or induce an immune response in the mammal as appropriate over a reasonable period of time. The amount of modified dendritic cells administered to the mammal is not critical inasmuch as any amount of DC-mediator-expressing modified cells will have some effect; desirably, however, enough modified dendritic cells will be administered to achieve the desired effect in a reasonable period of time. Such determinations are routinely made in the art of immunotherapy as a matter of routine general clinical development of cancer vaccines and the like.

If an antigen is to be administered to the mammal together with or separately from (e.g., sequentially) the modified DC-mediator-expressing dendritic cell, the antigen should be administered in a sufficient amount to realize induction of an immune response to the antigen. Desirably, the immune response is induced over a reasonable period of time. Here, again, while the amount of antigen administered to the mammal is not critical as even relatively small amounts of antigen will induce an immune response, desirably, enough antigen is administered to induce an immune response to the antigen over a reasonable period of time. However, not so much antigen should be administered as to adversely affect the health and overall well-being of the mammal. Such determinations are routinely made in the art of immunotherapy and vaccines as a matter of routine.

Similarly, if an antigen is to be contacted with a modified dendritic cell ex vivo, prior to the modified dendritic cell being administered to the mammal, the amount of antigen brought into contact with the modified dendritic cell(s) should be sufficient to activate or prime the dendritic cell. The amount of antigen used will depend, in part, on the immunogenicity of the antigen. Such determinations are also within the ordinary skill in the art.

In any event, consideration should be given to any adverse side affects, the overall health and well-being of the mammal, the age and body weight of the mammal, and the severity of any disease state, such as infection, cancer or a tumor. Adverse side effects should be kept to a reasonably tolerable level. Actual dosing and scheduling of dosages can vary, depending on intermammal differences in pharmacokinetics, drug distribution, metabolism and the like.

One skilled in the art can determine the appropriate dose, schedule, and method of administration for the formulation of the composition being used, in order to achieve the desired effective level in the mammal. When the present inventive method is used to suppress cancer or tumor growth, one skilled in the art also can readily determine and use an appropriate indicator of the effective level of the DC-mediator by a direct (e.g., cancer or tumor biopsy or radio-imaging of the cancer or tumor) or indirect (e.g., PSA levels in the blood) analysis of appropriate samples (e.g., blood and/or tissues).

Further, with respect to determining the effective level for suppression of cancer or tumor growth, suitable animal models are available and have been widely implemented for evaluating the in vivo efficacy against cancer of recombinant DNA protocols (see, e.g., PCR). These models include those of the Examples.

When used to suppress cancer or tumor growth, the pharmaceutical composition can contain other pharmaceuticals, in conjunction with a nucleic acid molecule comprising a nucleic acid sequence encoding a DC-mediator. In particular, it is contemplated that an anticancer or antitumor agent be employed, such as, preferably, a recombinant virus, a nucleic acid/liposomal formulation (or other nucleic acid delivery formulation), or another vector system (e.g., retrovirus or adenovirus), either as a viral particle or as a nucleic acid/liposomal formulation. Further representative examples of these additional pharmaceuticals that can be used in addition to those previously described, include chemotherapeutic agents, immunostimulants, antiviral compounds, and other agents and treatment regimes (including those recognized as alternative medicine) that can be employed to treat cancer. Anticancer compounds include, but are not limited to, angiostatin, endostatin, anti-HER-2/neu antibody, and tamoxifen. Immunomodulators and immunostimulants include, but are not limited to, various interleukins, cytokines, antibody preparations, and interferons.

In a further modification of the present invention, administering MIP-3α or SDF-1α (to the mammal attracts DC to the vicinity of administration in the mammal, thereby enhancing immunity in the mammal. MIP-3α and SDF-1α are chemo-attractants involved in directional migration of DC in vivo. These chemo-attractants can also be used to induce an immune response to a cancer or infectious disease involving administration of either MIP-3α or SDF-1α to a mammal having cancer or infected with a disease. After administration, a dendritic cell is then attracted to a cancerous area or infected area, thereby inducing an immune response to the cancer or infectious disease, respectively, in the mammal.

### EXAMPLES

The invention can be more clearly understood with reference to the following examples. The following examples further illustrate the present invention, but should not be construed as in any way limiting its scope.

### General Methods

Animals

Female C57B1/6 (H-2^{b}) and Balb/c (H-2^{d}) mice, 6-8 wks old, were obtained from the Jackson Laboratories (Bar Harbor, Me.), and housed under specific pathogen-free conditions.

Cell Culture

CT26 is an undifferentiated colon adenocarcinoma cell line (H-2^{d}) originally derived by intrarectal injections of N-nitroso-N-methylurethane in a female Balb/c mouse (provided by N. P. Restifo, National Cancer Institute, Bethesda, Md.). The SVBalb fibroblast cell line is also syngenic to Balb/c mice (provided by L. Gooding, Emory University, Atlanta, Ga.). CT26.CL25 is derived from CT26 cells modified to express the E. coli β-galactosidase (βgal) gene. C3 is a cell line originally derived by transfecting C57B1/6 mouse embryonal fibroblasts (H-2^{b}) with a plasmid containing the entire genome of the human papilloma virus type 16 (provided by C. J. M. Melief, University Hospital Leiden, The Netherlands). The CL7 fibroblast cell line (H-2^{d}), the B16 murine melanoma cell line (H-2^{b}), and the Lewis lung carcinoma cell line (H-2^{b}) were obtained from the American Type Culture Collection (Manassas, Va.).

The CT26 and C3 cell lines were maintained in complete RPMI-1640 medium (10% fetal bovine serum, 2 mM L-glutamine, 100 μg/ml streptomycin, and 100 U/ml penicillin; GIBCO BRL, Gaithersburg, Md.). The CT26.CL25 cell line was maintained in complete RPMI-1640 medium containing 400 μg/ml G418 (GIBCO BRL). DC were generated from mouse bone marrow precursors in complete RPMI-1640 medium with recombinant murine GM-CSF (100 U/ml; Sigma Chemical Co., St. Louis, Mo.) and recombinant murine interleukin-4 (IL-4; 2 ng/ml; R & D Systems, Minneapolis, Minn.) as described previously (Song et al., J. Exp. Med. 186: 1247 (1997)). All other cell lines were maintained in complete Dulbecco's minimum essential medium (DMEM) (GIBCO BRL).

Adenovirus Vectors

The adenovirus vectors used in these examples were based on the Ad5 backbone with deletions of E1 and E3 and the expression cassette in the E1 region. Briefly, the AdmCD40L is an E1-deleted, E3-deleted adenovirus vector that carries an expression cassette in the E1 position containing the CMV immediate early promoter/enhancer driving the cDNA for the murine CD40L (mCD40L). AdMIP-3α is also an E1-deleted, E3-deleted adenovirus vector that carries an expression cassette in the E1 position containing the CMV immediate early promoter/enhancer driving the cDNA for the human MIP-3α. Similarly, AdSDF-1α is an E1-deleted, E3-deleted adenovirus vector that carries an expression cassette in the E1 position containing the CMV immediate early promoter/enhancer driving the cDNA for the human SDF-1α. AdNull, used as a control vector in this study, is similar to AdmCD40L and AdMIP-3α, but without a gene in the expression cassette. Propagation, purification and titration of the adenovirus vectors were as previously described (Rosenfeld et al., Science 252: 431 (1991); Rosenfeld et al., Cell 68: 143 (1992)).

Pseudomonas Immunization

To immunize the mice, AdmCD40L-modified DC (moi 100, 4 hr, 37° C.) were incubated with heat killed (56° C., 1 hr) Pseudomonas (PAO1) for 4 hr at a ratio of 10 bacteria equivalents to one DC. The adenoviral vector was added first, immediately followed with the bacteria. Gentamicin sulfate (Sigma Chemical) was then added to a concentration of 200 μg/ml and the cell suspension was incubated for a further 30 min to kill the remaining bacteria. The cells were extensively washed twice with PBS, and 5×10⁴ DC in 100 μl PBS were injected intravenously in the tail vein.

Anti-Pseudomonas Antibodies

Anti-Pseudomonas antibodies were assessed in serum using a standard ELISA protocol. To assess the titer of respiratory mucosal anti-Pseudomonas antibodies, respiratory ELF was prepared by instilling 1.5 ml of PBS to mouse lungs and withdrawing the fluid. After centrifuging, the supernatant was collected and assayed for end-point titers of anti-Pseudomonas antibodies by ELISA.

Statistical Analysis

All data are reported as mean ± standard error. Statistical comparison was made using either Fisher's exact method or two-way analysis of variance (ANOVA), and a value of p<0.05 was accepted as indicating significance. Survival evaluation was carried out using Kaplan-Meir analysis.

### Example 1

This example demonstrates the modification of dendritic cells to produce CD40L and other DC-lymphocyte co-stimulatory molecules, such as IL-12 and MIP-1α.

DC from Balb/c mice were transduced with AdmCD40L (AdmCD40L-modified DC), AdNull (AdNull-modified DC), as a control, or phosphate-buffered saline (PBS) (i.e., no adenoviral vector), also as a control, at a multiplicity of infection (moi) of 40 for 4 hr, and plated on 24-well plates at 5×10⁶ cells/mi. Transduced DC were incubated with anti-mCD40L mAb MR1 (10 μg/ml; PharMingen, San Diego, Calif.) or the same amount of the control hamster IgG (PharMingen). After incubation for 72 hr at 37° C., the supernatant (400 μl) was harvested and centrifuged to remove debris. The levels of murine IL-12 or MIP-1α released into the culture medium were assessed by enzyme-linked immunosorbent assay (ELISA), using the mouse IL-12 p40 or MIP-1α immunoassay (R & D Systems), respectively.

AdmCD40L-modified DC enhanced expression of CD40L and other DC-lymphocyte co-stimulatory molecules. AdmCD40L-modified DC cultured at the standard density (2×10⁶ cells/ml) had a three- to six-fold increase in the number of DC expressing CD80 (B7-1) and CD54 (ICAM-1), as compared with AdNull-modified DC. When DC were cultured at lower density (2×10⁵ cells/ml) after AdmCD40L modification, DC expressed CD80⁺and CD54⁺, but at a slightly decreased level compared to DC cultured at a higher density, suggesting that at least some of the DC self-activation was via a bystander mechanism. In this regard, despite the similar percentage of CD40L-expressing DC cultured at higher or lower density, the lower-density culture was associated with a three- to four-fold decrease in the percentage of DC expressing CD80⁺CD40L⁻ (11.3% vs. 2.9%) or CD54⁺ CD40L⁻ (19.2% vs. 6.5%). DC cultured at a lower density expressed no fewer CD80⁺CD40L⁺ or CD54⁺CD40L⁺ than DC cultured at a higher density.

Expression of other surface molecules CD86 (B7-2) and CD48 (the mouse homologue of LFA-3) was also augmented in the higher density cultures by AdmCD40L modification (CD86⁺: AdNull (2.8%) vs. AdmCD40L (27.8%); CD48⁺: AdNull (17.6%) vs. AdmCD40L (27.1%)). CD25 (IL-2Rα), an activation marker of lymphocytes and macrophages, was expressed on only a small percentage of AdmCD40L-modified DC. A similar proportion of AdNull-modified DC expressed CD25. The percentages of cells expressing CD25 were <10% and were independent of AdmCD40L-mediated CD40L expression, indicating that there were minimal numbers of macrophages and lymphocytes contaminating the DC cultures.

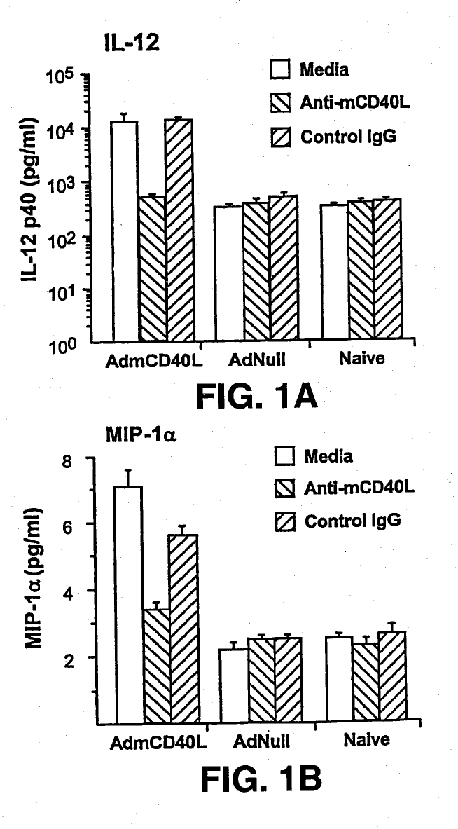

ELISA analyses, as shown in FIGS. 1A & B, which are bar graphs of IL-12 p40 (FIG. 1A) (pg/ml) and MIP-1α (FIG. 1B) (pg/ml) secreted by dendritic cells (DC) to which AdmCD40L, AdNull (control) or PBS (naive) was administered, confirmed that Ad vector-mediated administation of a DNA encoding CD40L to DC induced the DC to secrete cytokines. Infection of DC by AdmCD40L stimulated the production of IL-12 (approximately 10⁴ pg/ml; p<0.0001), whereas AdNull-infection did not (approximately 2.5 ×10² pg/ml; p>0.9). AdmCD40L, but not AdNull, infection of DC also induced MIP-1α secretion (p<0.0001). Both IL-12 and MIP-1α secretion were inhibited by addition of anti-mCD40L mAb MR1 as compared with control IgG (IL-12, p<0.0001; MIP-1α, p<0.01), indicating that the AdmCD40L vector-directed CD40L expression on the DC cell surface was responsible for stimulating the DC to secrete IL-12 and MIP-1α.

Thus, DC can be modified to produce CD40L and other DC-lymphocyte co-stimulatory molecules.

### Example 2

This example demonstrates in vivo production of CTL after administration of AdmCD40L-modified DC.

Balb/c mice bearing CT26-derived tumors were intratumorally inoculated with AdmCD40L- or AdNull-modified DC, or were not inoculated. Ten days after intratumoral injection of AdmCD40L-modified DC to 8 day established tumors, splenocytes were isolated from two mice, pooled, and restimulated for 5 days at 3×10⁶ or 4×10⁶ cells/ml with 10⁶ cells/ml CT26 or B16 cells treated with 100 μg/ml mitomycin C (Sigma). After restimulation, viable cells were collected and tested in a ⁵¹Cr-release assay for their ability to lyse CT26 or B16 cells. The percentage of specific ⁵¹Cr release was calculated as 100×[(experimental release−spontaneous release)/(maximal release−spontaneous release)].

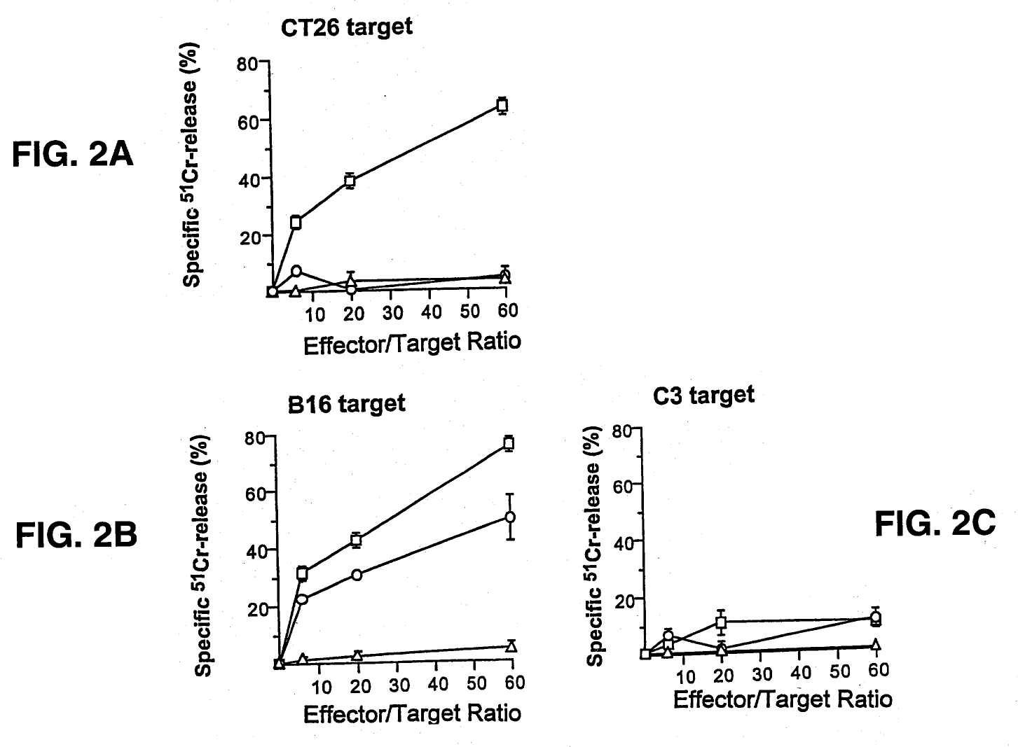

Direct injection of AdmCD40L-modified DC to CT26- or B16-derived tumors elicited tumor-specific CTL activity, as demonstrated in FIGS. 2A-C, which are graphs of the specific ⁵¹Cr release (%) vs. Effector/Target ratio. Cells from only mCD40L-DC treated mice exhibited specific lysis of CT26 target cells (FIG. 2A). C57B1/6 mice treated with mCD40L-modified DC exhibited a strong B16-specific splenic CTL response (FIGS. 2B & C). Controls for this analysis included lymphocytes obtained from tumor-bearing mice injected with AdNull-transduced DC or without any treatment. Splenocytes were restimulated with B16 cells as described above, and the resulting effector cells were evaluated for cytolytic activity against B16 and C3 cells. Injection with AdNull-transduced DC induced minimal CTL with reactivity against B16 cells, but AdmCD40L transduction markedly enhanced this specific cytolytic activity. No specific lysis with effector cells in the B16-derived model was observed against C3 cells.

This example demonstrates in vivo enhancement of immunity after administration of DC modified with an adenoviral vector comprising a DNA encoding CD40L.

### Example 3

This example demonstrates in vivo enhancement of immunity and suppression of tumor growth after administration of DC modified to express CD40L.

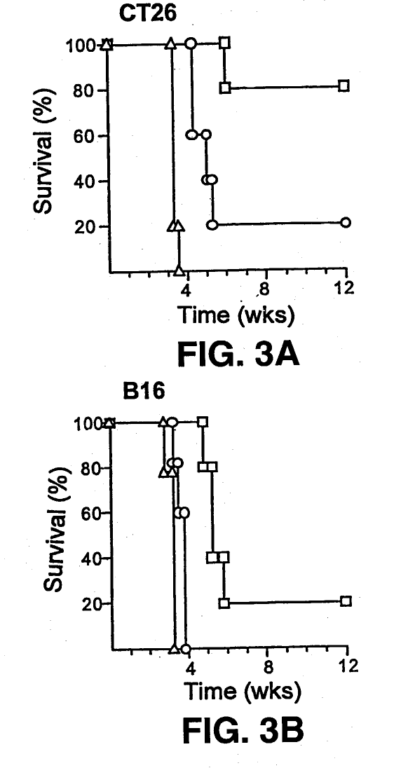

Adoptive transfer of splenocytes protected against a subsequent challenge with the identical tumor cells, as demonstrated in FIGS. 3A & B, which are graphs of survival (%) as a function of time (wks). In this context, adoptive transfer of 5×10⁷ splenocytes isolated 10 days after intratumoral administration of AdmCD40L- or AdNull-modified DC mediated 80% or 20% protection against a CT26-derived challenge over a 12 wk period, respectively (AdmCD40L-DC compared to naive control, p<0.005; AdmCD40L-DC compared to AdNull-DC, p<0.05; FIG. 3A). Splenocytes from B16-derived tumor-bearing mice treated with AdmCD40L-modified DC provided 20% protection (compared to naive, p<0.005; FIG. 3B). In contrast, transfer of lymphocytes isolated from mice injected with AdNull-modified DC mediated only a minor enhancement in survival compared to no infusion of splenocytes (p<0.05; AdmCD40L-DC vs. AdNull-DC, p<0.0005).

This example demonstrates in vivo enhancement of immunity and subsequent survival after administration of DC modified in vitro by administration of an adenoviral vector comprising a DNA encoding CD40L.

### Example 4

This example demonstrates in vivo enhancement of immunity and suppression of tumor growth after administration of DC modified to express CD40L.

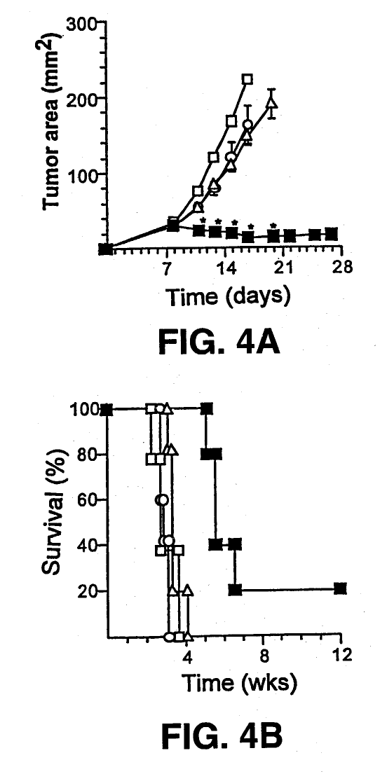

Antitumor CTL activity was also associated with a systemic therapeutic effect in a distant two-tumor model of metastatic disease, as demonstrated in FIG. 4A, which is a graph of tumor area (mm²) as a function of time (days), and FIG. 4B, which is a graph of survival (%) as a function of time (wks). Mice bearing bilateral B16-derived flank tumors were treated by intratumoral injection to the left tumor with CD40L-modified DC. This therapy actively suppressed growth of the untreated right tumor as well as the treated tumor, with 20% of the mice alive at the end of the experiment on day 84 with no detectable tumors in both flanks. In contrast, tumors grew progressively and were eventually lethal in control mice treated with AdNull-infected DC or without any therapy. As a further control, administration of CD40L-modified DC to a Lewis lung carcinoma-derived tumor in the left flank of mice had no beneficial effect on inhibiting B16-derived tumor growth in the right flank of mice, confirming the tumor specificity of the antitumor effect.

This example demonstrates that a systemic therapeutic effect can be realized upon administration of DC modified to express CD40L.

### Example 5

This example demonstrates that administration of DC modified to express CD40L can suppress tumor growth in vivo.

Tumor cells (5×10⁵ B16 or 2×10⁵ CT26) were injected subcutaneously in the right flank of mice. On day 8, mice were inoculated into the tumor with 100 μl of DC or CL7 cells, infected with the AdmCD40L or AdNull vector (moi 40, 24 hr) or mock-infected. The size of each tumor was assessed three times weekly and recorded as the average tumor area (mm²) ± standard error by measuring the largest perpendicular diameters. When animals became moribund or the tumors reached 15 mm in diameter, the mice were sacrificed and this was recorded as the date of death for survival studies. For some studies, where indicated, mice were challenged in both flanks with tumor cells: 5×10⁵ B16 in the right flank, and 5x10⁵ B16 or LLC in the left flank.

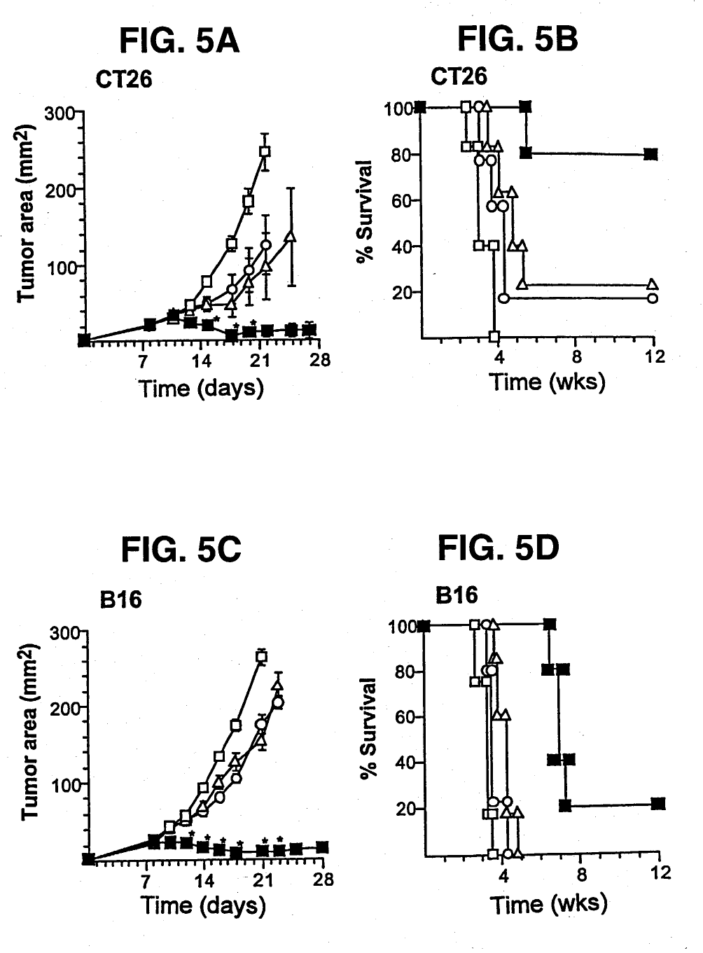

Treatment of CT26-derived tumor-bearing Balb/c mice (H-2^{d}) with 2×10⁶ CD40L-modified DC induced significant inhibition of tumor growth (p<0.05) at time points 15 to 20 days, and resulted in long-term survival in most mice (p<0.05), as demonstrated in FIG. 5A, which is a graph of tumor area (mm³) as a function of time (days), and FIG. 5B, which is a graph of survival (%) as a function of time (wks). Administration of 2×10⁶ AdNull- or mock-infected DC also had some beneficial effect as compared to no treatment (tumor size days 15 to 22, p<0.05; survival, p<0.05).

In the B16-derived tumor model in C57B1/6 mice (H-2^{b}), tumor growth was suppressed significantly by 2×10⁶ CD40L-modified DC treatment as compared to that of all other control groups (days 12 to 23, p<0.005), resulting in survival advantage (p<0.005), as demonstrated in FIG. 5C, which is a graph of tumor area (mm³) as a function of time (days) and FIG. 5D, which is a graph of survival (%) as a function of time (wks). To a lesser extent, the B16-derived tumor treated with 2×10⁶ AdNull- or mock-infected DC was also suppressed significantly as compared to that without any treatment (tumor area days 14 to 21, p<0.005; survival, p<0.05).

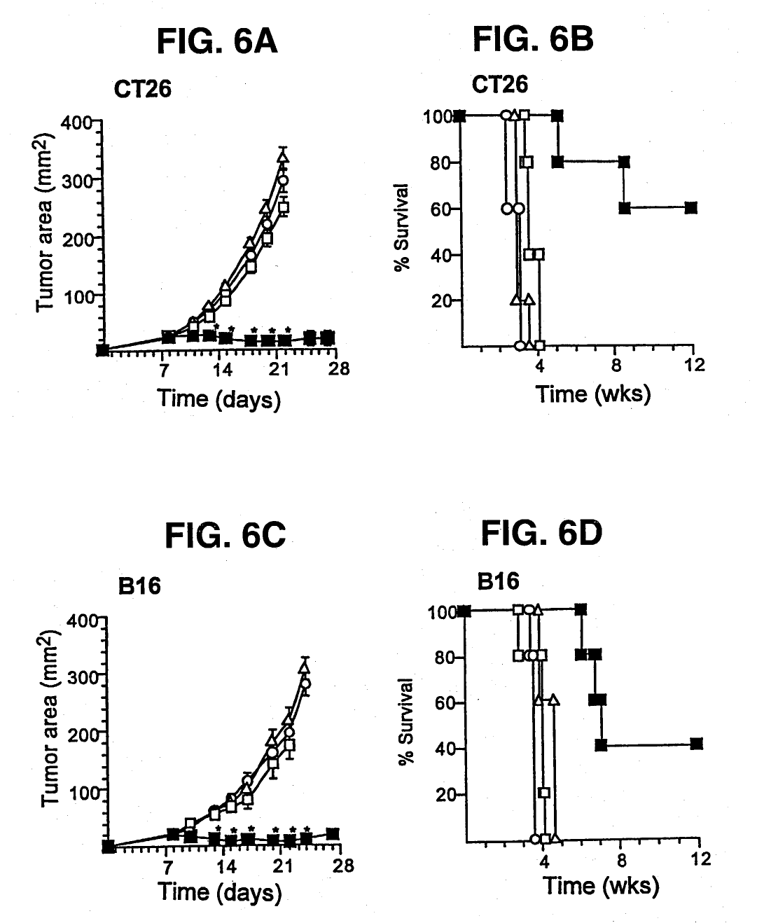

Marked tumor suppression was also observed with administration to the tumors of one-tenth the numbers (2×10⁵) of AdmCD40L-infected DC. In this context, 2×10⁵ AdmCD40L-DC suppressed the growth of established tumors (both CT26-derived and B16) when injected intratumorally, but AdNull- or mock-infected DC did not. Balb/c mice bearing CT26-derived 8-day established tumors were treated by direct injection with 2×10⁵ AdmCD40L-modified DC. This therapy significantly inhibited tumor growth days 13 to 22 (p<0.0001) and survival at 12 wk in 60% (p<0.005) in contrast with AdNull- or mock-infected DC as well as no treatment, as demonstrated in FIG. 6A, which is a graph of tumor area (mm³) as a function of time (days) and FIG. 6B, which is a graph of survival (%) as a function of time (wks). Intratumoral injection with AdNull- or mock-infected DC had no therapeutic effect on CT26-derived tumor-bearing mice days 11 to 22 (p>0.7). Similar results were achieved in the B16-derived established tumors. The growth of B16-derived tumors injected with AdmCD40L-modified DC was suppressed significantly over days 13 to 24 (p<0.0005) with enhanced survival (p<0.005), whereas tumors injected with AdNull- or mock-infected DC grew in a similar fashion to naive tumors from days 10 to 22 (p>0.2) and did not have enhanced survival (p>0.2), as demonstrated in FIG. 6C, which is a graph of tumor area (mm³) as a function of time (days) and FIG. 6D, which is a graph of survival (%) as a function of time (wks).

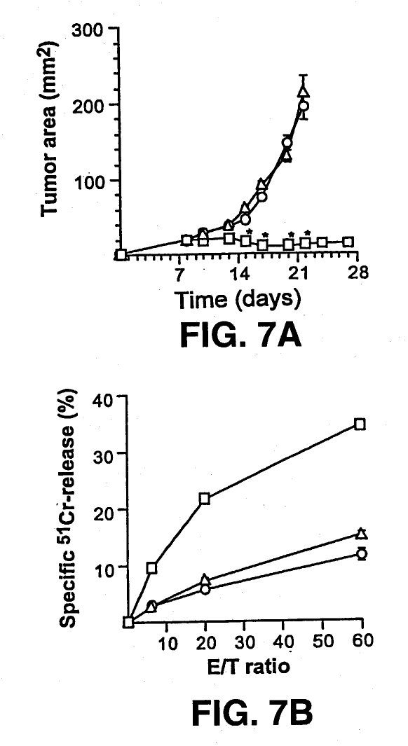

When injected intratumorally, CD40L-modified DC induced therapeutic tumor immunity, but CD40L-modified fibroblasts did not, as demonstrated in FIG. 7A, which is a graph of tumor area (mm²) as a function of time (days) and FIG. 7B, which is a graph of specific ⁵¹Cr-release (%) as a function of Effector/Target ratio. Growth of subcutaneous CT26-derived tumors was profoundly affected by 2×10⁶ AdmCD40L-modified DC (FIG. 5A), whereas tumors treated with 2×10⁶ of CL7 fibroblasts that had been infected with AdmCD40L at the identical moi grew as did the control group without any treatment (p>0.3; FIG. 7A). This antitumor effect in vivo correlated to tumor-specific CTL activity demonstrated by splenocytes from mice treated with the same regimen (FIG. 7B). Animals bearing untreated CT26-derived tumors generated a 15% lysis of ⁵¹Cr-labeled CT26 target cells at an Effector/Target ratio of 60/1. This lytic activity was enhanced to 34% after CD40L-modified DC treatment, but not after intratumoral injection of CD40L-modified CL7 cells. Taken together with the adoptive transfer and spleen mRNA and immunohistochemical data (FIGS. 6, 8, 10), these data indicate that optimal therapeutic immunity of CD40L-modified DC depends, at least in part, not on regional stimulation by CD40L expression in tumors, but on migration of activated DC to the lymphoid tissues to stimulate tumor antigen-specific T cells.

This example demonstrates that administration of DC modified to produce CD40L to a mammal having a tumor significantly suppresses tumor growth and increases long-term survival.

### Example 6

This example demonstrates that both the MIP-3α protein and the SDF-1α protein can attract DC in vitro.

Primary bone marrow DC was obtained from mouse bone marrow precursors (Inaba et al, 176 J.Exp.Med. 1693 (1992)). In brief, lymphocyte- and erythrocyte-depleted murine bone marrow cells harvested from femurs were plated in complete RPMI medium supplemented with recombinant murine GM-CSF (100 U/ml) and recombinant murine interleukin 4 (20 ng/ml; Genzyme, Farmington, Mass.). On days 2 and 4, nonadherent granulocytes were gently removed and fresh medium was added. On day 6, loosely adherent proliferating DC aggregates were dislodged and replated. On day 6 of culture, nonadherent cells with the typical morphological features of DC were suspended at a 6 concentration of 10⁶ cells/ml in RPMI medium 1640 (GIBCO BRL, Gaithersburg, Md.) supplemented with 1% fetal bovine serum (GIBCO BRL).

Fifty μl of suspension were placed in the upper chamber, and 25 μl of supernatant of A549 cells infected for 3 days with AdMIP-3α, AdSDF-1α, or AdNull (control), uninfected (naive) cells were placed in the lower chamber. The chamber was incubated for 90 min at 37° C. Directed migration was expressed as the number of cells seen in 5 hpf that had migrated to the lower chamber. Checkerboard analysis of the supernatnants of AdMIP-3α- and AdSDF-1α-infected A549 cells was carried out to distinguish chemotaxis from chemokinesis. Different dilutions of supernatants were added to upper and lower chambers and the apparatus was incubated for 90 min at 37° C. Directed migration was expressed as the number of cells seen in hpf that had migrated to the lower chamber.

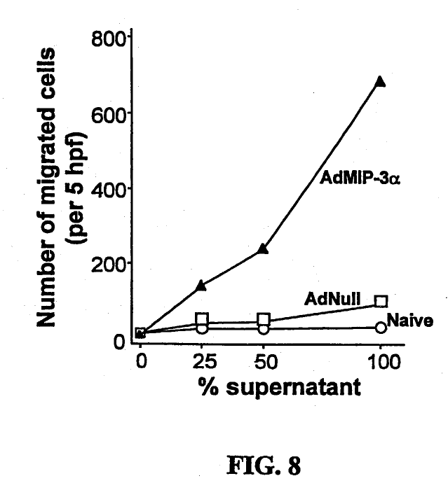

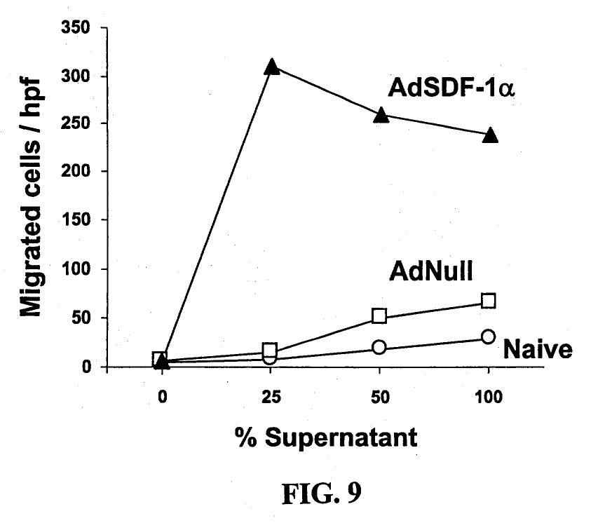

To confirm the biological activity of the secreted human MIP-3α and SDF-1α, chemotaxis for DC was assessed, as demonstrated in FIG. 8 for MIP-3α, which is a graph of the number of migrated cells (per 5 hpf) as a function of the % supernatant, and FIG. 9 for SDF-1α, which is a graph of the number of migrated cells (per hpf) as a function of the % supernatant. The supernatant of A549 cells infected with AdMIP-3α or AdSDF-1α showed markedly increased chemotactic activity for DC compared with controls. Checkerboard analysis demonstrated that the migration of DC induced by the AdMIP-3α supernatant and the AdSDF-1α supernatant was due to the stimulation of directed migration (chemotaxis) toward the attractant, rather than simply an increase in random motility (chemokinesis; see, Table 1 (AdMIP-3α); Table 2 (AdSDF-1α)).

TABLE 1

Upper Chamber (%)

| Lower Chamber (%) | 0 | 25 | 50 | 100 |
|---|---|---|---|---|
| 0 | 10 | 14 | 12 | 9 |
| 25 | 140 | 42 | 33 | 14 |
| 50 | 240 | 90 | 40 | 19 |
| 100 | 691 | 466 | 157 | 24 |

TABLE 2

Upper Chamber (%)

| Lower Chamber (%) | 0 | 25 | 50 | 100 |
|---|---|---|---|---|
| 0 | 4 | 4 | 5 | 4 |
| 25 | 321 | 73 | 26 | 10 |
| 50 | 267 | 178 | 103 | 17 |
| 100 | 242 | 131 | 133 | 53 |

This example demonstrates that cells modified to produce a DC-mediator can attract DC in vitro.

### Example 7

This example demonstrates that the AdMIP-3α protein and the AdSDF-1α protein can attract DC in vivo.

Three days after intradermal injection of AdMIP-3α, AdSDF-1α AdNull, PBS or control (5×10⁸ pfu in 20 μl), C57B1/6 mice were sacrificed and the skin harvested. Cryostat sections (8 μm) were placed on the slides, air-dried, and fixed in acetone for 10 min and air-dried for at least 30 min. After washing in PBS/0.01% Triton X 100, the slides were incubated with PBS/0.01% Triton X 100/1% normal goat serum for 60 min, then incubated overnight at 4° C. with a 1:200 dilution of rat anti-mouse dendritic cell antibody (anti-DEC205, NLDC145; Serotec, Washington, D.C.) or rat anti-mouse isotype-matched control IgG2a (Serotec). To identify T cells, anti-mouse CD8a mAb (1Y-2; PharMingen), anti-mouse CD4 (L3T4; PharMingen) and control rat IgG2a, K isotype standard (PharMingen) were utilized. After washing in PBS/0.01% Triton X 100, the slides were incubated with a 1:200 dilution of goat anti-rat IgG (Oregon Green antibody; Molecular Probe, Eugene, Oreg.), and the slides were examined using a fluorescence microscope.

The MIP-3α mRNA was detected only in the skin injected with AdMIP-30α, and the SDF-1α mRNA was detected only in the skin injected with AdSDF-1α. Fluorescence microscopy demonstrated the accumulation of dendritic cells in the AdMIP-3α- and AdSDF-1α-infected skin as assessed with the DEC205 polyclonal antibody, but not in controls.

This example demonstrates that cells modified to produce a DC-mediator can attract DC in vivo.

### Example 8

This example demonstrates administration of AdMIP-3α and AdSDF-1α to tumors in vivo.

B16 tumor cells (3×10⁵ cells) and CT26 tumor cell (3×10⁵ cells) were administered subcutaneously to C57B1/6 mice and Balb/c mice, respectively. After 8 days, the B16-derived tumors were injected with AdMIP-3α (5×10⁸ pfu in 100 μl), AdSDF-1α (3×10⁵ pfu in 100 μl), AdNull (5×10⁸ pfu in 100 μl) or PBS (100 μl) and the CT26-derived tumors were injected with AdMIP-3α (5×10⁸ pfu in 100 μl), AdNull (5×10⁸ pfu in 100 μl) or PBS (100 μl). To demonstrate expression of the AdMIP-3α or the AdSDF-1α in the tumors, Northern analysis was carried out, as described above, with RNA extracted from tumors 3 days after intratumoral administration and hybridized (20 μg/lane) with a human MIP-3α probe, a human SDF-1α probe, or a GAPDH probe. DC and T cells attracted to the tumors 3 days after administration of the AdMIP-3α, AdSDF-1α, AdNull, PBS or controls were assessed by immunohistochemistry, as described above.

The MIP-3α mRNA was detected only in B16- and CT26-derived tumors injected with AdMIP-3α, while the SDF-1α mRNA was detected only in B16-derived tumors injected with AdSDF-1α. Fluorescence microscopy showed the accumulation of dendritic cells in B16- and CT26-derived subcutaneous tumors induced by the AdMIP-3α vector, and the accumulation of dendritic cells in B16-derived subcutaneous tumors induced by the AdSDF-1α vector, but not in controls.

To demonstrate AdMIP-3α and AdSDF-1α modifications of tumor growth in vivo, mice were injected subcutaneously on day 0 with tumor cells (3×10⁵) including: CT26.CL25 (n=30 mice), CT26 (n=30), B16 (n=30) and Lewis lung cell carcinoma (n=30). All injections were performed into the shaved right flank in a total volume of 100 μl. When the tumors had grown to 15 to 25 mm² (day 6 for CT26.CL25; day 8 for CT26, B16 and LLC), mice were inoculated into the tumors with 100 μl of the AdMIP-3α, AdSDF-1α (3×10⁵ cells) or AdNull vectors (5×10⁸ pfu) in PBS or PBS alone (100 μl) for CT26, B16 and LLC, while CT26.CL25 was only inoculated with 100 μl of the AdMIP-3α or AdNull vectors (5×10⁸ pfu) in PBS or PBS alone (100 μl). The size of each tumor was monitored three times weekly. The tumor area was calculated, and expressed as the average tumor area (mm²) ± standard error. If animals appeared moribund or the diameter of the tumors reached 20 mm, the mice were sacrificed and this was recorded as the date of death for survival studies. Survival of the animals was assessed using standard methodology.

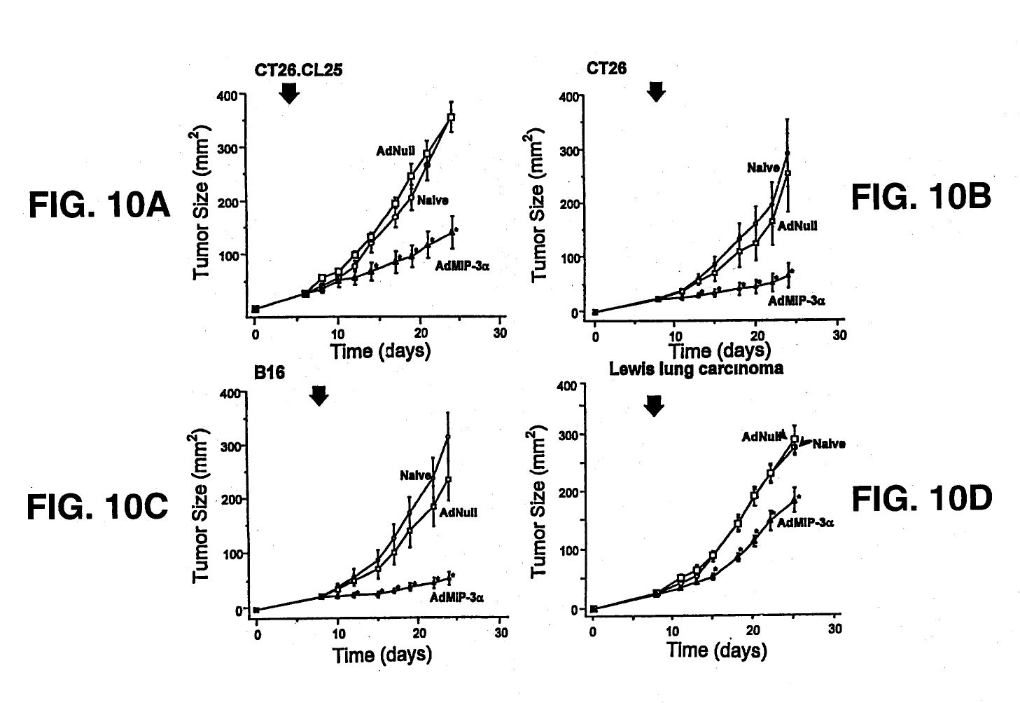

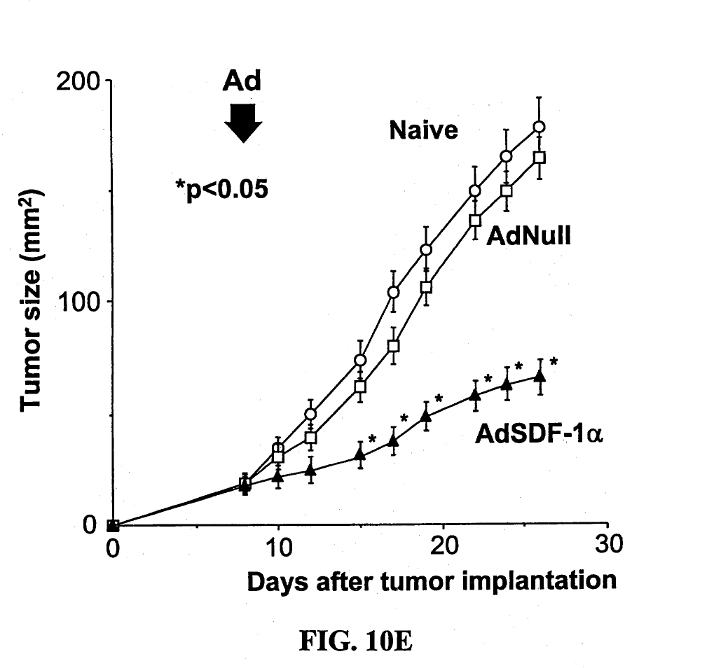

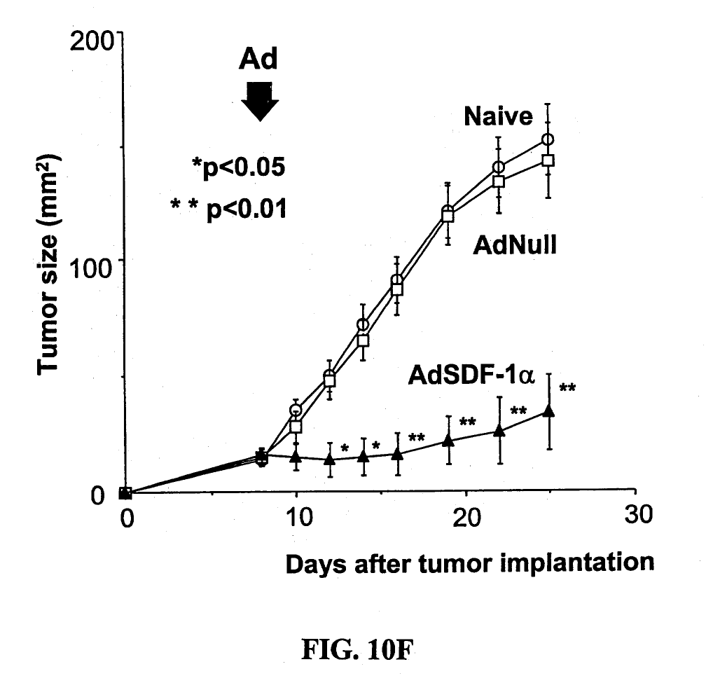

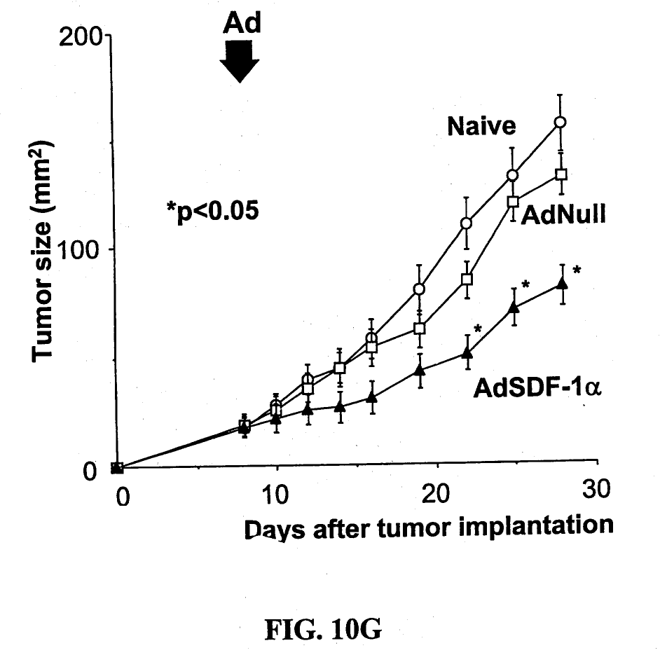

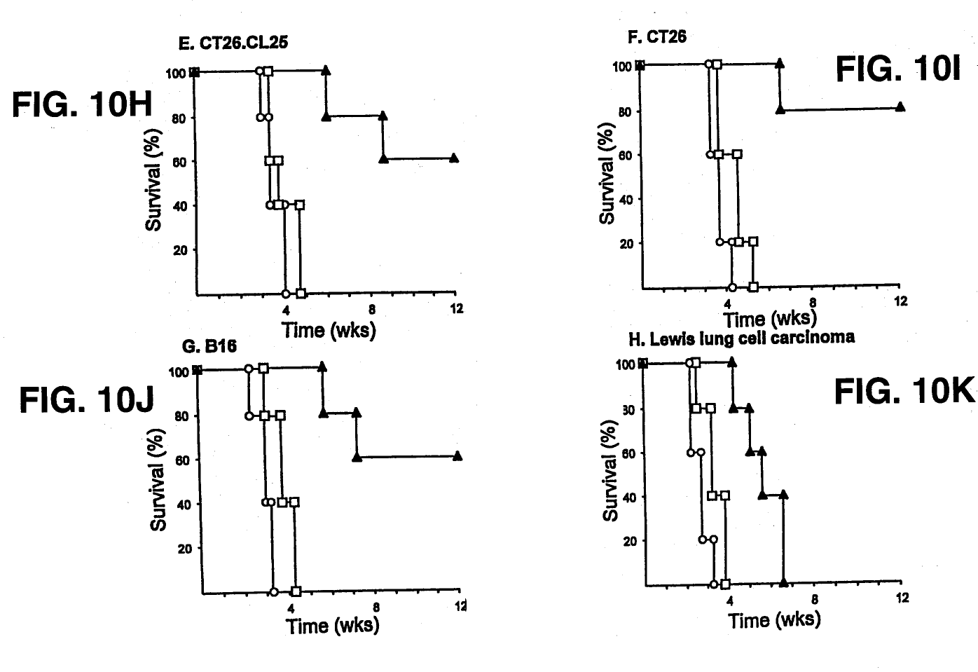

Intratumoral injection of AdMIP-3α and AdSDF-1α inhibited the growth of the different murine tumors, as demonstrated in FIGS. 10A-G, which are graphs of tumor size (mm²) as a function of time (days) and FIGS. 10H-K, which are graphs of survival (%) as a function of time (wks). In the CT26.CL25-derived tumor model in Balb/c mice (H-2^{d}), treatment with AdMIP-3α induced significant inhibition of tumor growth (p<0.05) and there was significant long-term survival, whereas administration of AdNull had no beneficial effect (FIGS. 10A & H). In the CT26-derived tumor model in Balb/c mice, tumor size of AdMIP-3α-treated mice also regressed significantly as compared to that of control groups (p<0.05), and there was significant long term survival (p<0.05; FIGS. 10B & I), while tumor size of AdSDF-1α-treated mice also regressed significantly as compared to that of control groups (p<0.05; FIG. 10E). In the B16-derived tumor model in C57B1/6 mice (H-2^{b}), tumor growth was also suppressed significantly by AdMIP-3α (p<0.05), and there was significant long-term survival (p <0.05; FIGS. 10C & J), while tumor growth was also suppressed significantly by AdSDF-1α (p<0.05; FIG. 10F). In the less immunogenic Lewis lung carcinoma model in C57B1/6 mice, tumor size was also inhibited significantly by AdMIP-3α although to a lesser degree than the other models (p<0.05), but there was no enhanced survival (p >0. 1, FIGS. 10D & K), while tumor size was also inhibited significantly by AdSDF-1α, although again to a lesser degree than the other models (p<0.05; FIG. 10G).

This example demonstrates modification of cells to produce a DC-mediator and subsequent suppression of tumor growth in vivo.

### Example 9

This example demonstrates the induction of a tumor-specific immune response after intratumoral injection of AdMIP-3α.

Splenocytes were isolated 12 days after Ad vector injection into the tumors (as described above) and restimulated at 3×10⁶ cells/ml with 10⁶ cells/ml irradiated (5000 rad) tumor cells. After 5 days of culture, the in vitro restimulated splenocytes were quantified using a ⁵¹Cr-release assay for their ability to lyse tumor cells. The percentage of specific a ⁵¹Cr release was expressed as follows: 100×(experimental release−spontaneous release)/ (maximal release−spontaneous release). SV Balb and C3 cells were used as control for Balb/c- and C57B1/6-syngenic tumors, respectively.

To demonstrate that in vivo administration of the AdMIP-3α vector sensitized the cellular host defense system against the relevant tumor, ten days after the inoculation of the four types of tumors with Ad vectors (as described above), the spleens were removed. Splenocytes (3×10⁷ cells/mouse) were injected into recipient animals by tail vein. Seven days later (day 0), recipient animals were challenged by subcutaneous injection in the right flank with 3×10⁵ relevant tumor cells. Survival was assessed as described above.

Intratumoral administration of AdMIP-3α was associated with accumulation of mostly CD8a-positive T cells infiltrating CT26-derived subcutaneous tumors. For example, when CT26 colon carcinoma cells growing in Balb/c mice were injected with AdMIP-3α, CD8a-positive cells were significantly increased, with lesser numbers of CD4-positive cells evident. Similar results were observed with the CT26-, B16- and Lewis lung carcinoma-derived tumors.

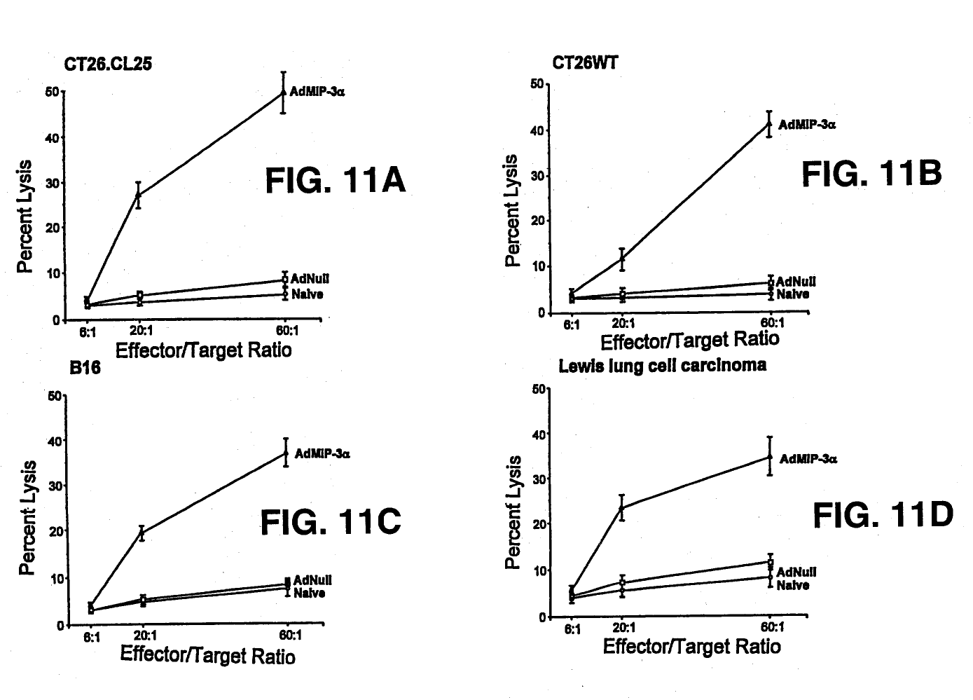

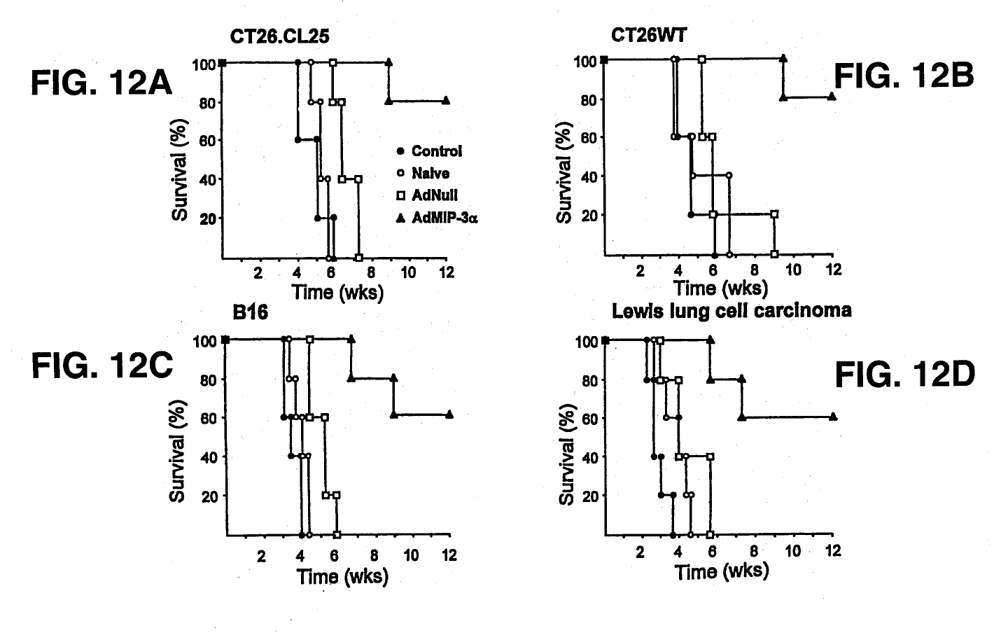

Transduction with AdMIP-3α elicited tumor-specific CTL activity in all four tumor models, as demonstrated in FIGS. 11A-D, which are graphs of percent lysis as a function of Effector/Target ratio. Balb/c mice bearing CT26.CL25-derived tumors were intratumorally inoculated with AdMIP-3α or AdNull. Effector cells generated from splenocytes 12 days after the innoculation by culture with irradiated CT26.CL25 tumor cells exhibited specific lysis of CT26.CL25 target cells in cells obtained only from AdMIP-3α treated animals (FIG. 10A). No apparent lysis was observed against irrelevant but syngenic fibroblast SVBalb cells. In animals with CT26-, B16- and Lewis lung cell carcinoma-derived tumors, effector cells from only AdMIP-3α-treated mice also exhibited specific lysis of relevant target cells (FIGS. 11B-D). Evidence that in vitro specific cytolysis was relevant in vivo came from studies demonstrating that adoptive transfer of splenocytes protected against a subsequent challenge with the identical tumor cells in all four tumor models (p<0.05), as demonstrated in FIGS. 12A-D, which are graphs of survival (%) as a function of time (wks).

This example demonstrates in vitro and in vivo enhancement of immunity after administration of an adenoviral vector comprising a DNA encoding a DC-mediator.

### Example 10

This example demonstrates the ability of AdmCD40L-modified DC pulsed with P. aeruginosa to induce naive B cells to secrete P. aeruginosa-specific antibodies in vitro in the absence of CD4⁺ T cells.

CD19⁺ B lymphocytes (10⁵/ml), purified (magnetic cell sorter system; Miltenyi Biotech, Auburn, Calif.) from a naive C57B1/6 mouse, were cultured for 14 days in a final volume of 200 μl with 10⁵/ml AdmCD40L-modified DC (as described previously) pulsed with heat-killed P. aeruginosa (AdmCD40L+PA; 10 Pseudomonas per DC; PAO1 strain of Pseudomonas, provided by A. Prince, Columbia University), AdNull-modified DC (as described previously) pulsed with heat-killed P. aeruginosa (AdNull+PA), AdmCD40L-modified DC (AdmCD40L), or DC with no treatment in the presence of IL-4 (20 ng/ml; R&D Systems) in a 96-well plate. To demonstrate in vitro processing was involved, AdmCD40L-modified DC pulsed with heat-killed P. aeruginosa were treated with brefeldin A (5 μg/ml), cytochalasin D (10 μg/ml), or ammonium chloride (NH₄Cl; 50 mM; all from Sigma Chemical, St. Louis, Mo.) for 30 min prior to, as well as during, the Pseudomonas pulse. To demonstrate CD4⁺ T cell independence, where indicated, both DC and B cells were prepared from CD4−/− mice, or CD11c (α_{X} integrin)+DC were purified from the DC culture with the MACS system (Miltenyi Biotech) before modifying DC with AdmCD40L and Pseudomonas and subsequent co-culture with naive B cells.

After 14 days, the titer of various isotypes of P. aeruginosa-specific antibodies in culture supernatants (200 μl) was determined by ELISA. Briefly, flat-bottomed, 96-well plates (Bio-Rad Laboratories, Hercules, Calif.) were coated at 4° C. with 10⁷ cfu heat-killed P. aeruginosa in 0.05 M carbonate buffer pH 9.6 (Sigma Chemical) with 0.2% sodium azide. The coating solution was discarded, and 1% bovine serum albumin in PBS was added for 30 min. After discarding the blocking solution, serial dilutions of samples were incubated at 23° C. for 1 hr. The plates were washed with the washing buffer (0.05% Tween 20 in PBS), and rabbit anti-mouse subtype (IgM, IgG1, IgG2a, IgG2b, IgG3 or IgA) specific IgG were added (Bio-Rad). The plates were incubated at 23° C. for 1 hr, rinsed with the washing buffer, and incubated with diluted goat anti-rabbit IgG horseradish peroxidase-conjugated antibodies (Bio-Rad) at 23° C., 1 hr. After washing out unreacted conjugated antibodies, the plates were developed with peroxidase substrate solution (Bio-Rad) at 23° C. for 30 min, and then assessed in an ELISA reader at 415 nm. End-point titers were determined as the reciprocal of the dilution at or below a fixed absorbance value of 0.1, and any negative results were given a titer of the lowest dilution.

As controls for the specificity of the ELISA, no significant anti-Pseudomonas IgM, IgG, or IgA were detected in anti-sera obtained from mice immunized against E. coli, but positive anti-Pseudomonas IgM, IgG, and IgA were detected in sera from mice immunized against Pseudomonas. No other isotypes of P. aeruginosa-specific antibody were detected even at the lowest dilution. AdmCD40L-modified DC pulsed with P. aeruginosa processed and presented Pseudomonas antigens to cocultured B cells, resulting in the stimulation of IgM and IgA production specific for P. aeruginosa in the absence of CD4⁺ T cells.

The possibility that surface binding of extracellular P. aeruginosa during the coincubation with DC was responsible for the presentation of the Pseudomonas antigens was excluded using three types of pharmacologic inhibitors on antigen processing pathways, including brefeldin A (inhibition of ER/Golgi transport), cytochalasin D (suppression of actin-dependent phagocytosis), and ammonium chloride (inhibition of acid pH-dependent degradation). Pseudomonas-specific IgM and IgA secretion in the coculture of B cells and AdmCD40L-modified DC pulsed with P. aeruginosa was significantly abrogated when DC were primed with Pseudomonas in the presence of any one of these inhibitors (IgM p<0.0001; IgA p<0.0001). Although these inhibitors blocked the processing of the bacterial antigens for presentation in the DC, the DC modified with AdmCD40L still expressed CD40L following treatment with brefeldin A, cytochalasin D, or ammonium chloride (i.e., those treatments did not adversely modify the DC).

To eliminate the possibility that CD4⁺ T cells contaminating the DC or B cell preparations could be responsible for the observation of in vitro generation of Pseudomonas-specific antibodies, DC and B cells prepared from CD4−/− mice were used in similar coculture experiments. Despite this absolutely CD4-deficient condition, the results obtained for Pseudomonas-specific IgM and IgA production from B cells cocultured with AdmCD40L-modified DC pulsed with Pseudomonas were similar to that observed with components from wild-type mice. Consistent with this observation, MACS-sorted CD11c⁺DC purified from the DC culture, modified with AdmCD40L and pulsed with P. aeruginosa also induced Pseudomonas-specific IgM and IgA secretion from B cells.

This example, therefore, demonstrates the interaction between DC and B cells in the induction of an immune response in accordance with the present invention.

### Example 11

This example demonstrates the activation of B cells in vivo by CD40L-modified DC pulsed with P. aeruginosa.

C57B1/6 mice were immunized with AdmCD40L-modified DC (described previously) pulsed with heat-killed P. aeruginosa (AdmCD40L+PA), AdNull-modified DC (described previously) pulsed with heat-killed P. aeruginosa (AdNull+PA), or AdmCD40L-modified DC alone (AdmCD40L) at 5×10⁴ DC per mouse. Controls included naive mice without any immunization (“no immunization”). Two weeks after immunization, the titer of each isotype of P. aeruginosa-specific antibody was determined by ELISA (described previously). Other isotypes of anti-Pseudomonas antibodies (IgM, IgG1, IgG2a, IgG2b and IgG3) were assessed in epithelial lining fluid, but not detected in any group.

C57B1/6 mice immunized with AdmCD40L-modified DC pulsed with P. aeruginosa produced significant amounts of serum anti-Pseudomonas antibodies (IgM end-point titers 3-7 fold, p<0.0001; IgG1 3-4 fold, p<0.0001; IgG2b 2-4 fold, p<0.0001; and IgG3 3-5 fold, p<0.02), compared with mice immunized with AdNull-modified DC pulsed with P. aeruginosa, AdmCD40L-modified DC alone or nonimmunized mice. There was an insignificant increase in IgG2a levels (p>0.2).

As a control for the specificity of the anti-Pseudomonas antibodies detected in vivo, serum of mice immunized with AdmCD40L-modified DC pulsed with E. coli was negative for anti-Pseudomonas IgM, IgG, and IgA. Like the increase in anti-Pseudomonas IgM and IgG serum antibodies, there was a significant increase in serum IgA levels in the AdmCD40L+PA immunized animals compared to controls (p<0.0001). Interestingly, there was no significant difference of respiratory mucosal IgA anti-Pseudomonas antibodies in epithelial lining fluid in the AdmCD40L+PA immunized animals (p>0.1, all comparisons), except for the small difference between AdmCD40L-modified DC pulsed with P. aeruginosa and AdmCD40L-modified DC alone (p<0.05). No other isotypes of anti-P. aeruginosa antibodies were detected in ELF.

Thus, this example demonstrates the induction of an immune response by DC modified to express a dendritic cell-mediator and pulsed with antigen in accordance with the present invention.

### Example 12

This example demonstrates that CD40L-modified DC pulsed with heat-killed P. aeruginosa induced protective immunity, lasting at least 3 months, against a lethal challenge with Pseudomonas in vivo.

Immunization of C57B1/6 mice with 5×10⁴AdmCD40L-modified DC (described previously) pulsed with heat-killed P. aeruginosa 3 wk before a lethal challenge with 2×10⁵ cfu of P. aeruginosa enmeshed in agar beads resulted in beneficial survival in 90% of mice (Kaplan-Meier analysis, p<0.0005 to all other groups). In contrast, immunization with 5×10⁴ AdNull-modified DC (described previously) pulsed with heat-killed P. aeruginosa or 5×10⁴ AdmCD40L-modified DC alone, or no immunization led to <10% survival of the infected mice.

Similar results were achieved with mice immunized 3 months before the Pseudomonas challenge; 80% of mice immunized with AdmCD40L-modified DC pulsed with heat-killed P. aeruginosa survived at least 14 days after the lethal challenge with P. aeruginosa (Kaplan-Meier analysis, p<0.0001 to all other groups). In contrast, the control groups of mice receiving AdNull-modified DC pulsed with heat-killed P. aeruginosa or AdmCD40L-modified DC alone 3 months before the instillation died within 5 days.

This example demonstrates that DC modified to express a dendritic cell-mediator and then pulsed with an antigen induced an immune response and provided protection upon challenge, thereby providing an efficacious vaccine.

### Example 13

This example demonstrates that DC modified to express CD40L and pulsed with P. aeruginosa or E. coli induced protective immunity from P. aeruginosa or E. coli, respectively.

Groups of C57B1/6 mice received vaccinations composed of 5×10⁴ AdmCD40L-modified DC pulsed with either heat-killed P. aeruginosa or E. coli, or no vaccination. After 3 weeks, the mice were challenged with intratracheal administration of 2×10⁵ cfu of P. aeruginosa or 10⁸ cfu of E. coli.

Animals receiving immunization of P. aeruginosa-pulsed CD40L-activated DC were protected from Pseudomonas challenge, but immunization with E. coli-pulsed CD40L-activated DC did not protect the mice from Pseudomonas challenge, nor did no immunization (Kaplan-Meier analysis, p<0.0001, CD40L-activated DC pulsed with heat-killed P. aeruginosa compared with all other groups). In contrast, 60% of animals following immunization of E. coli-pulsed CD40L-activated DC were protected against E. coli challenge, whereas all nonimmunized animals or animals immunized with P. aeruginosa-pulsed CD40L-activated DC died following E. coli infection (Kaplan-Meier analysis, p<0.0005, DC pulsed with heat-killed E. coli compared with all other groups).

This example demonstrates successful vaccination through DC modified with a dendritic cell-mediator and exposed to an antigen.

### Example 14

This example demonstrates that anti-Pseudomonas immunity induced by CD40L-modified DC pulsed with Pseudomonas requires B cells, but not CD4⁺ T cells.

Markedly enhanced immunity against Pseudomonas induced by CD40L-modified DC pulsed with Pseudomonas could be achieved in the absence of CD4 T cells, i.e., CD40L genetic modification of DC not only augmented the anti-Pseudomonas immunity, but also affected the antigen-specific immunity independent of T cell help. In this context, studies with knockout mice demonstrated that B cells were required for anti-Pseudomonas immunity induced by AdmCD40L+PA vaccination (described previously), but CD4⁺ T cells were not required.

To evaluate the contribution of lymphocyte subpopulations to the protective immunity afforded by AdmCD40L+PA immunization, groups of CD4+ T cell-deficient, B cell-deficient or wild-type mice were immunized with or without AdmCD40L-modified DC (described previously) pulsed with P. aeruginosa, then challenged 3 wk later by intratracheal injection of P. aeruginosa. CD4−/− immunized mice were completely protected from lethal challenge with P. aeruginosa, as were wild-type immunized mice. In marked contrast, no protective immunity was observed in B cell-deficient immunized mice compared to wild-type mice without immunization; all B cell-deficient mice died within 3 days after Pseudomonas instillation (Kaplan-Meier analysis, p<0.001 between CD4+ T cell-deficient and B cell-deficient mice).

This example thus demonstrates that B cells, but not CD4+ T cells, are required for induction of an immune response and protection upon challenge in the methods of the present invention.

All of the references cited herein, including patents, patent applications, and publications, are hereby incorporated in their entireties by reference.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed is:

1. A method of enhancing immunity in a mammal, which method comprises modifying a dendritic cell in the mammal to produce a dendritic cell-mediator by transducing the dendritic cell directly with a replication-deficient adenoviral vector comprising a nucleic acid molecule that encodes the dendritic cell-mediator, wherein the dendritic cell-mediator is a cytokine, and whereupon the dendritic cell-mediator up-regulates dendritic cells, thereby enhancing immunity in the mammal.

2. The method of claim 1, wherein the dendritic cell-mediator increases dendritic cell maturation.

3. The method of claim 2, wherein the dendritic cell-mediator is a protein that binds to CD40 on the surface of the dendritic cell.

4. The method of claim 1, wherein the dendritic cell-mediator increases dendritic cell migration.

5. The method of claim 4, wherein the dendritic cell-mediator is a chemokine.

6. The method of claim 5, wherein the chemokine is macrophage inflammatory protein-3α.

7. The method of claim 1, wherein the mammal has a cancer and enhancing immunity in the mammal enhances an immune response to the cancer in the mammal.

8. The method of claim 1, wherein the mammal has an infectious disease and enhancing immunity in the mammal enhances an immune response to the infectious disease in the mammal.

9. A method of enhancing immunity in a mammal, which method comprises removing a dendritic cell from the mammal, modifying the dendritic cell to produce a dendritic cell-mediator by transducing the dendritic cell with a replication-deficient adenoviral vector comprising a nucleic acid molecule that encodes the dendritic cell-mediator, wherein the dendritic cell-mediator is a cytokine, and administering the modified dendritic cell to the mammal, whereupon the dendritic cell-mediator up-regulates dendritic cells, thereby enhancing immunity in the mammal.

10. A method of enhancing immunity in a mammal, which method comprises administering to the mammal a dendritic cell, which has been modified to produce a dendritic cell-mediator by transducing the dendritic cell with a replication-deficient adenoviral vector comprising a nucleic acid molecule that encodes the dendritic cell-mediator, wherein the dendritic cell mediator is a cytokine, and whereupon the dendritic cell-mediator up-regulates dendritic cells, thereby enhancing immunity in the mammal.

11. The method of claim 10, wherein the dendritic cell-mediator increases dendritic cell maturation.

12. The method of claim 11, wherein the dendritic cell-mediator is a protein that binds to CD40 on the surface of the dendritic cell.

13. The method of claim 10, wherein the dendritic cell-mediator increases dendritic cell migration.

14. The method of claim 13, wherein the dendritic cell-mediator is a chemokine.

15. The method of claim 14, wherein the chemokine is macrophage inflammatory protein-3α.

16. The method of claim 10, wherein the mammal has a cancer and enhancing immunity in the mammal enhances an immune response to the cancer in the mammal.

17. The method of claim 10, wherein the mammal has an infectious disease and enhancing immunity in the mammal enhances an immune response to the infectious disease in the mammal.

18. A method of inducing an immune response to an antigen in a mammal, which method comprises administering the antigen to a mammal, which mammal has been treated in accordance with the method of claim 1, whereupon an immune response to the antigen is induced.

19. A method of inducing an immune response to a cancer in a mammal, which method comprises administering an antigen of the cancer to the mammal, which mammal has been treated in accordance with the method of claim 7, whereupon an immune response to the cancer is induced.

20. The method of claim 19, wherein the antigen of the cancer is a cell of the cancer.

21. A method of inducing an immune response to an infectious disease in a mammal, which method comprises administering an antigen of a causative agent of the infectious disease to the mammal, which has been treated in accordance with the method of claim 8, whereupon an immune response to the infectious disease is induced.

22. The method of claim 18, wherein the antigen is encoded by a nucleic acid molecule.

23. The method of claim 22, wherein the nucleic acid molecule comprising a nucleic acid sequence encoding a dendritic cell-mediator also encodes the antigen.

24. A method of inducing an immune response to an antigen in a mammal, which method comprises administering the antigen to a mammal, which has been treated in accordance with the method of claim 9, whereupon an immune response to the antigen is induced.

25. A method of inducing an immune response to an antigen in a mammal, which method comprises administering a dendritic cell to a mammal in accordance with the method of claim 10, which method further comprises contacting the dendritic cell, which has been modified to produce a dendritic cell-mediator by transducing the dendritic cell with a replication-deficient adenoviral vector comprising a nucleic acid molecule that encodes the dendritic cell-mediator, with the antigen prior to administration of the dendritic cell to the mammal, whereupon the dendritic cell-mediator up-regulates dendritic cells and is a cytokine, and whereupon an immune response to the antigen is induced in the mammal.

26. A method of inducing an immune response to a cancer in a mammal, which method comprises administering an antigen of the cancer to a mammal, which has been treated in accordance with the method of claim 16, whereupon an immune response to the cancer is induced.

27. A method of inducing an immune response to an infectious disease in a mammal, which method comprises administering an antigen of a causative agent of the infectious disease to a mammal, which has been treated in accordance with the method of claim 17, whereupon an immune response to the infectious disease is induced.

28. The method of claim 25, wherein the antigen is encoded by a nucleic acid molecule.

29. The method of claim 28, wherein the nucleic acid molecule comprising a nucleic acid sequence encoding a dendritic cell-mediator also encodes the antigen.

30. A method of inducing an immune response to a cancer in a mammal, which method comprises administering a dendritic cell to a mammal in accordance with the method of claim 16, which method further comprises contacting the dendritic cell, which has been modified to produce a dendritic cell-mediator by transducing the dendritic cell with a replication-deficient adenoviral vector comprising a nucleic acid molecule that encodes the dendritic cell-mediator, with an antigen of the cancer prior to administration of the dendritic cell to the mammal, whereupon the dendritic cell-mediator up-regulates dendritic cells and is a cytokine, and whereupon an immune response to the cancer is induced.

31. A method of inducing an immune response to an infectious disease in a mammal, which method comprises administering a dendritic cell to a mammal in accordance with the method of claim 17, which method further comprises contacting the dendritic cell, which has been removed from the mammal and modified to produce a dendritic cell-mediator by transducing the dendritic cell with a replication-deficient adenoviral vector comprising a nucleic acid molecule that encodes the dendritic cell-mediator, with an antigen of a causative agent of the infectious disease prior to administration of the dendritic cell to the mammal, whereupon the dendritic cell-mediator up-regulates dendritic cells and is a cytokine, and whereupon an immune response to the infectious disease is induced.

32. The method of claim 31, wherein the antigen is encoded by a nucleic acid molecule comprising a nucleic acid sequence.

33. The method of claim 32, wherein the nucleic acid molecule comprising a nucleic acid sequence encoding a dendritic cell-mediator also encodes the antigen.

34. A method of treatment comprising the method of claim 1.

35. A method of treatment comprising the method of claim 10.

36. The method of claim 2, wherein the dendritic cell-mediator is tumor necrosis factor-α.

37. The method of claim 5, wherein the chemokine is macrophage-derived chemokine.

38. The method of claim 5, wherein the dendritic cell mediator is stromal cell-derived factor 1-α.

39. The method of claim 11, wherein the dendritic cell-mediator is tumor necrosis factor-α.

40. The method of claim 14, wherein the chemokine is macrophage-derived chemokine.

41. The method of claim 14, wherein the chemokine is stromal cell-derived factor-lax.

42. The method of claim 1, wherein the replication-deficient adenoviral vector is administered to the mammal, and wherein the replication-deficient adenoviral vector comprises a bi-specific molecule or adenoviral coat protein that binds to dendritic cells in the mammal.
